SEQUENCE LISTING (15 sequences)

SEQ ID NO: 1 - protein, 4 aa, Unknown
  Description of Unknown ′KDEL′ motif derived from unknown organism
KDEL

SEQ ID NO: 2 - DNA, 22 bp, Artificial Sequence
  Description of Artificial Sequence Synthetic primer
ggtgggtttgagtttggaggtt

SEQ ID NO: 3 - DNA, 22 bp, Artificial Sequence
  Description of Artificial Sequence Synthetic primer
ggggaaacgctctatgaacagg

SEQ ID NO: 4 - DNA, 33 bp, Artificial Sequence
  Description of Artificial Sequence Synthetic primer
cccgggcgccaccatggaggtggtggacgagac

SEQ ID NO: 5 - DNA, 28 bp, Artificial Sequence
  Description of Artificial Sequence Synthetic primer
ctcgagggaggcagctcagtcacacagg

SEQ ID NO: 6 - DNA, 28 bp, Artificial Sequence
  Description of Artificial Sequence Synthetic primer
ctcgaggaggtggtggacgagaccgaag

SEQ ID NO: 7 - DNA, 28 bp, Artificial Sequence
  Description of Artificial Sequence Synthetic primer
ctcgagggaggcagctcagtcacacagg

SEQ ID NO: 8 - protein, 11 aa, Artificial Sequence
  Description of Artificial Sequence Synthetic peptide
MEQKLISEEDL

SEQ ID NO: 9 - DNA, 22 bp, Artificial Sequence
  Description of Artificial Sequence Synthetic primer
gggagggggcttcaaggagtgt

SEQ ID NO: 10 - DNA, 22 bp, Artificial Sequence
  Description of Artificial Sequence Synthetic primer
cccccagcatgccgatgtacac

SEQ ID NO: 11 - DNA, 21 bp, Artificial Sequence
  Description of Artificial Sequence Synthetic primer
cctttcgccagggggatcttg

SEQ ID NO: 12 - DNA, 24 bp, Artificial Sequence
  Description of Artificial Sequence Synthetic primer
gctaagtgccttctctacacctgc

SEQ ID NO: 13 - DNA, 22 bp, Artificial Sequence
  Description of Artificial Sequence Synthetic primer
ggaaaatgcttctgtccgtttg

SEQ ID NO: 14 - protein, 1139 aa, Mus musculus
MEVVDETEALQRFFEGHDISGALEPSNIDTSILEEYIGKEDASDLCFPEISAPASTASFPHGPPAIPGSSGLHHLSPPGSGPSPGRHGPLPPPTYGTPLNCNNNNGMGTAPKPFLGGSGPPIKAEPKAPYAPGTLPDSPPDSGSEAYSPQQVNDPHLLRTITPETLCHVGVSSRLEHPPPPPAHLPGPPPPPPPPPHYPVLQRDLYMKAEPPVPPYAAMGPGLVPPELHHTQQTQVLHQLLQQHGAELPPHPSKKRKHSESPPNTLNAQMLNGMIKQEPGTVTALPPHPARAPSPPWPPQGPLSPGTGSLPLSIARAQTPPWHPPGAPSPGLLQDSDSLSGSYLDPNYQSIKWQPHQQNKWATLYDANYKELPMLTYRVDADKGFNFSVGDDAFVCQKKNHFQVTVYIGMLGEPKYVKTPEGLKPLDCFYLKLHGVKLEALNQSINIEQSQSDRSKRPFNPVTVNLPPEQVTKVTVGRLHFSETTANNMRKKGKPNPDQRYFMLVVALQAHAQNQNYTLAAQISERIIVRASNPGQFESDSDVLWQRAQLPDTVFHHGRVGINTDRPDEALVVHGNVKVMGSLMHPSDLRAKEHVQEVDTTEQLKRISRMRLVHYRYKPEFAASAGIEATAPETGVIAQEVKEILPEAVKDTGDVVFANGKTIENFLVVNKERIFMENVGAVKELCKLTDNLETRIDELERWSHKLAKLRRLDSLKSTGSSGAFSHAGSQFSRAGSVPHKKRPPKLANKSSPAVPDQACISQRFLQGTIIALVVVMAFSVVSMSTLYVLSLRSEEDLVDADGSLAVSTSCLLALLRPQDPGGSEAMCPCRSSQSFGTTQLRQSSMTTGLPGTQPSLLLVTKSASGPALRALDLCSSQPCPIVCCSPPVSSPATDPALGPTLTPTPSPSSNPKHSGPGQMAPLPVTNIRAKSWGISANGISYSKHSKSLEPLASPVVPFPGGQSKTKNSPSFNLQSRARRGAPQPSPSPAQFTQTQGQLDPAPSLTSIQLLENSMPITSQYCVPEGACRLGNFTYHIPVSSSTPLHLSLTLQMNSSTPVSVVLCSLTSEEEPCEEGGFLQRFHPHQDTQGTSHQWPVTILSFREFTYHFRVTLLGQANCSSEAIVQPATDYYFHFYRLCD

SEQ ID NO: 15 - protein, 1111 aa, Homo sapiens
MHWLPAGHDINGALEPSNIDTSILEEYISKEDASDLCFPDISAPASSASYSHGQPAMPGSSGVHHLSPPGGGPSPGRHGPLPPPGYGTPLNCNNNNGMGAAPKPFPGGTGPPIKAEPKAPYAPGTLPDSPPDSGSEAYSPQQVNEPHLLRTITPETLCHVGVPSRLEHPPPPPAHLPGPPPPPPPPPHYPVLQRDLYMKAEPPIPHYAAMGQGLVPTDLHHTQQSQMLHQLLQQHGAELPTHPSKKRKHSESPPSTLNAQMLNGMIKQEPGTVTALPLHPTRAPSPPWPPQGPLSPGPGSLPLSIARVQTPPWHPPGAPSPGLLQDSDSLSGSYLDPNYQSIKWQPHQQNKWATLYDANYKELPMLTYRVDADKGFNFSVGDDAFVCQKKNHFQVTVYIGMLGEPKYVKTPEGLKPLDCFYLKLHGVKLEALNQSINIEQSQSDRSKRPFNPVTVNLPPEQVTKVTVGRLHFSETTANNMRKKGKPNPDQRYFMLVVALQAHAQNQNYTLAAQISERIIVRASNPGQFESDSDVLWQRAQVPDTVFHHGRVGINTDRPDEALVVHGNVKVMGSLMHPSDLRAKEHVQEVDTTEQLKRISRMRLVHYRYKPEFAASAGIEATAPETGVIAQEVKEILPEAVKDTGDMVFANGKTIENFLVVNKERIFMENVGAVKELCKLTDNLETRIDELERWSHKLAKLRRLDSLKSTGSSGAFSHAGSQFSRAGSVPHKKRPPKVASKSSSVVPDQACISQRFLQGTIIALVVVMAFSVVSMSTLYVLSLRTEEDLVDTDGRSSQSFGTTQLRQSPLTTGLPGIQPSLLLVTTSLTSSAPGSAVRTLDMCSSHPCPVICCSSPTTNPTTGPSLGPSFNPGHVLSPSPSPSTNRSGPSQMALLPVTNIRAKSWGLSVNGIGHSKHHKSLEPLASPAVPFPGGQGKAKNSPSLGFHGRARRGALQSSVGPAEPTWAQGQSEPVPSLTSIQVLENSMSITSQYCAPGDACRPGNFTYHIPVSSGTPLHLSLTLQMNSSSPVSVVLCSLRSKEEPCEEGSLPQSLHTHQDTQGTSHRWPITILSFREFTYHFRVALLGQANCSSEALAQPATDYHFHFYRLCD

(19) United States
(12) Patent Application Publication
(10) Pub. No.: US 2011/0179502 A1
(43) Pub. Date: Jul. 21, 2011

(54) MODULATION OF GM98 (MRF) IN REMYELINATION

(76) Inventors: Ben Emery, Palo Alto, CA (US); Ben A. Barres, Palo Alto, CA (US)

(21) Appl. No.: 13/003,834

(22) PCT Filed: Jul. 16, 2009
(86) PCT No.: PCT/US09/04155

§ 371 Date: Mar. 30, 2011

Related U.S. Application Data
(60) Provisional application No. 61/081,279, filed on Jul. 16, 2008.
(60) Provisional application No. 61/120,307, filed on Dec. 5, 2008.

(51) Int. Cl. G01N 33/00; C07H 21/00; A61K 38/02; A61K 39/395; A61K 31/713; A61P 25/00; C12N 15/63; A01K 67/00; C12Q 1/02; C12N 5/10; A61K 49/00; C12Q 1/68

(52) U.S. Cl. 800/3; 536/23.1; 514/1.1; 424/172.1; 514/44 A; 435/320.1; 800/13; 435/29; 435/325; 424/9.2; 435/6.13

(57) ABSTRACT

The present invention provides compositions and methods for regulating remyelination and promoting oligodendrocyte differentiation by modulating GM98 (also known as MRF) expression and activity. Compositions and methods for treating neuropathies and screening for bioactive agents are also provided herein.

### CROSS REFERENCE

This application claims the benefit of U.S. Provisional application Ser. No. 61/081,279, filed Jul. 16, 2008, and Ser. No. 61/120,307, filed Dec. 5, 2008, both of which are incorporated herein in their entirety.

This invention was made with government support under RO1 EY10257 from the National Eye Institute. The government has certain rights in the invention.

### BACKGROUND OF THE INVENTION

Multiple sclerosis (MS) is an inflammatory demyelinating disease of the central nervous system (CNS) with clinical deficits ranging from relapsing-remitting to chronic-progressive patterns of expression. Although the etiology of MS is unknown, autoreactive CD4⁺ T cell responses mediate inflammatory damage against myelin and oligodendrocytes. (Bruck et al., J. Neurol. Sci. 206, 181-185 (2003)). CNS lesions have focal areas of myelin damage and are also associated with axonal pathology, neural distress, and astroglial scar formation (Compston et al., Lancet 359, 1221-1231 (2002)). Clinical presentation includes various neurological dysfunctions including blindness, paralysis, loss of sensation, as well as coordination and cognitive deficits.

Damage or injury to myelin has severe consequences on conduction velocity and the vulnerability of neurons to axonal destruction. There is a correlation between axon loss and progressive clinical disability and intact myelin is important in the maintenance of axonal integrity (Dubois-Dalcq et al., Neuron 48, 9-12 (2005)). Spontaneous remyelination occurs during the early phases of human MS, however, persistent CNS inflammation and the failure of myelin repair during later stages of the disease ultimately lead to permanent debilitation.

Mature oligodendrocytes (OLs) are responsible for remyelination. Thus, the failure of remyelination is typically associated with deficiencies in the generation of mature oligodendrocytes, their ability to myelinate, and/or neurons that are unreceptive to myelination. In demyelinating diseases such as multiple sclerosis, surviving OLs and their progenitors (oligodendrocyte precursor cells, or OPCs) are often found in and around within demyelinated regions. The failure of these surviving cells to remyelinate nearby axons may reflect an inability of OPCs to differentiate or for the postmitotic oligodendrocytes to re-initiate the expression of a set of genes required for myelination. Myelination relies on the coordination of multiple signals including those that precisely localize oligodendrocytes and their precursors (Tsai et al., Cell 110:373-383 (2002); Tsai et al., J. Neurosci. 26:1913-22. (2006)), regulate appropriate cell numbers (Banes et al., Cell 70:31-46 (1992); Calver et al., Neuron 20:869-882 (1998)), and mediate interactions between oligodendrocytes and their target axons (Sherman and Brophy, Nat Rev Neurosci. 6:683-690 (2005)), and thus deficiencies in any of these processes can contribute to the failure in remyelination.

There is a need to develop effective methods for enhancing and promoting myelination or remyelination. Strategies that promote either the differentiation of OPCs, the progenitor pools, or re-initiation of myelination by existing postmitotic oligodendrocytes can be beneficial in establishing remyelination. The present invention provides compositions and methods directed to promoting remyelination. The findings disclosed herein demonstrates expression of genes involved in myelin production is affected by GM98 (Gene Model 98), also known as MRF (Myelin gene Regulatory Factor), and myelination.

### SUMMARY OF THE INVENTION

The present invention provides methods and compositions for modulating MRF expression and/or activity. The expression and activity of genes regulated by MRF are also described herein. The methods and compositions can be used to promote remyelination, screen for bioactive agents that modulate MRF expression/activity or the genes it regulates, as well as for the treatment of neuropathies.

One aspect of the invention is an isolated nucleic acid molecule comprising a cell type specific expression regulatory element operably linked to a nucleic acid sequence encoding MRF or a functional variant thereof. Furthermore, the cell type specific regulatory element is an inducible or constitutive promoter. The present invention also provides a vector comprising the nucleic acids described herein, and host cells type comprise the vectors and nucleic acids of the present invention.

Also provided herein is a transgenic animal comprising a MRF transgene. The transgene can comprise the nucleic acid sequences described herein. Furthermore, the transgene can comprise mutations and deletions, such as deletion of an exon. The exon can be an exon in the putative DNA binding domain, such as exon 8. The can also be flanked by recombinase sites, such as sites for Cre or Flp. The transgenic animal can also comprise a recombinase transgene, such as Cre recombinase or Flp. The transgenes can also be operably linked to a cell type specific expression regulatory element. The transgenic animal can be a mammal, such as a mouse or rat.

Specific expression of the nucleic acid or transgenes can be in a neural cell, such as a glial cell. The glial cell can be an oligodendrocyte, oligodendrocyte precursor, Schwann cell, astrocyte, or microglial cell. The cell type specific regulatory element can be from a CC1, myelin basic protein (MBP), ceramide galactosyltransferase (CGT), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), GFAP, AQP4, PDGFa, RG5, pGlycoprotein, neurturin (NRTN), artemin (ARTN), persephin (PSPN), sulfatide or proteolipid protein (PLP), Olig1, or Olig2 gene.

The transgenic animal can also be used in screening for a candidate bioactive agent effective in promoting remyelination/myelination. The method can comprise administering a candidate bioactive agent to the animal and assaying for an increase in the expression level of at least one gene in Table 1, in comparison to a control animal, wherein the increase is indicative of said bioactive agent promoting remyelination in the animal; and/or, observing a change in myelination in the animal in comparison to a control animal. The bioactive agent can be a peptide, antibody, aptamer, siRNA, miRNA, EGS, antisense molecule, peptidomimetic, or small molecule. In another aspect of the present invention, a method for screening a candidate bioactive agent effective in promoting remyelination/myelination in an animal is provided, wherein the method comprises administering a candidate bioactive agent to an animal; and, assaying for an increase in the expression level of MRF in comparison to a control animal, wherein the increase is indicative of the bioactive agent promoting myelination in the animal. The present invention also provides methods of screening for a candidate bioactive agent effective in modulating MRF activity. In one aspect, the method comprises contacting a test cell with a candidate bioactive agent; and, assaying for a change in the expression level of MRF in comparison to a control cell.

In yet another aspect of the present invention, a composition for treating a neuropathy in a subject comprising a bioactive agent that modulates MRF activity in said subject is provided. The compositions can promote remyelination/myelination in a subject. The compositions can promote stem cells or embryonic stem cells to differentiate into oligodendrocytes. The compositions can comprise a first bioactive agent, such as MRF or an agent modulates MRF activity or expression, and a second bioactive agent that induces oligodendrocyte differentiation. For example, the second bioactive agent can promote Sox10, Nxk2.2, Olig1, and/or Olig2. The second bioactive agent can be Sox 10, Nxk2.2, Olig1, or Olig2. Compositions can comprise MRF, Sox10, Nxk2.2, Olig1, Olig2, or a combination thereof. The composition can be used to treat a neuropathy such as a demyelinating condition, such as multiple sclerosis. The compositions provided herein can be used in methods of treating a neuropathy in a subject, wherein the method comprises administering to the subject a therapeutically effective amount of a bioactive agent that modulates MRF activity. The method can promote remyelination/myelination in a subject. Administration can comprise administering a first bioactive agent, such as MRF, or a bioactive agent that modulates MRF expression or activity, and a second bioactive agent, wherein the second bioactive agent also induces oligodendrocyte differentiation. Administering the second bioactive agent can be prior to, concurrent with, or subsequent to administering the first bioactive agent. The second bioactive agent can promote the activity of Sox10, Nxk2.2, Olig1, Olig2 or a combination thereof. The second bioactive agent can have a synergistic effect.

### BRIEF DESCRIPTION OF THE DRAWINGS

The novel features of the present invention are set forth with particularity in the appended claims. A better understanding of the features and advantages of the present invention is obtained by reference to the following detailed description that sets forth illustrative embodiments, in which the principles of the invention are utilized, and the accompanying drawings of which:

FIG. 1 depicts the identification of an OL-specific transcript GM98/MRF within the CNS. A) Expression levels of GM98/MRF in acutely isolated cells from the CNS was determined by Affymetrix analysis (probe set 1439506_at). B) Northern blot confirming expression of MRF as a single ˜5.5 Kb transcript within P20 brain and cultured oligodendrocytes (OLs), but not heart tissue, mirroring the expression pattern of the established OL marker CNP 1. GAPDH is shown as a control for mRNA levels. C) In situ hybridization of the established OL marker PLP and MRF within sagittal P16 brain sections. Both genes show a clear white-matter expression pattern. D) Higher magnification images of PLP and MRF expression within the lateral corpus callosum showing identical distribution of labeled cells.

FIG. 2 depicts the peptide structure and subcellular localization of MRF. A) Comparison of the peptide sequence of the mouse MRF and human C11Orf9 genes. B) Diagram showing structural regions of the mouse MRF and human C11Orf9 proteins and their homology within these regions. C-E) Subcellular localization of myc-tagged MRF within HEK cells. Staining of Myc-MRF transfected HEK cells with anti-myc (E) indicates a strong nuclear localization of the protein, with essentially complete co-localization with the nuclear counterstain DAPI in transfected cells. Cells transfected with a non myc-tagged MRF (D) and a myc-tagged protein displayed a cytoplasmic localization (C) providing a negative control for the staining and a comparison, respectively.

FIG. 3 illustrates expression of MRF is important for OL maturation. A) Live:Dead assay showing viability and morphology of OLs transfected with siCont or siMRF for 24-96 hours during differentiation (viable cells stained with Calcien AM in the green channel, non-viable nuclei stained with Ethidium Homodimer in the red channel). Cells transfected with siMRF showed less deposition of membrane sheets and decreased viability relative to siCont transfected cells. B) Viability of siMRF transfected cells relative to siCont transfected cells 24-96 hours after transfer to differentiating conditions. siMRF transfected cells displayed a significant reduction in viability relative to siCont transfected cells from 48 hours post differentiation. **P<0.01. C) Expression of the OPC marker NG2 and the early-OL marker MBP in of OLs transfected with siCont or siMRF at 24, 48, and 96 hours differentiation. Although siMRF transfected cells down-regulate NG2 expression with a similar temporal profile to siCont transfected cells, they displayed a delayed and reduced induction of MBP expression. D) Quantification of the proportion of siCont and siMRF transfected OLs expressing MBP from 24-96 hours differentiation. siMRF transfected cells displayed a reduced proportion of MRF expression at all time points. **P<0.01. E) Expression of the late-OL marker MOG in of OLs transfected with siCont or siMRF for 24-96 hours during differentiation. siMRF transfected cells displayed a reduced induction of MOG expression. F) Quantification of the proportion of siCont and siMRF transfected OLs expressing MOG from 24-96 hours differentiation. siMRF transfected cells displayed a reduced proportion of MRF expression from 48 h differentiation. **P<0.01. Results are representative of 3 independent experiments.

FIG. 4 depicts analysis of OL gene expression with MRF knockdown. A) Northern blot analysis of gene expression in cells transfected with siCont or siMRF as OPCs then cultured for 48 hours in differentiating conditions. RNA from brain, heart and cultured astrocyte samples were provided for positive and negative controls, respectively. Transfection of cells with siMRF strongly reduced the amount of MRF transcript present, also resulting in a clear inhibition in the expression of PLP, and, to a lesser extent, CNP 1. Northern results for GAPDH and visualization of the 18S ribosomal bands are provided as loading controls. B) Results of Affymetrix analysis of gene expression in OPCs and cells transfected with siCont or siMRF as OPCs then cultured for 48 hours in differentiating conditions, showing expression levels of selected OPC markers OPC markers NG2, PDGFRCt and Ki67. The down-regulation of these OPC markers was not affected by MRF knockdown. C) Expression of pan-OL lineage marker Sox10, early-OL markers (Ugt8, CNP 1, PLP1, MBP) and late-OL markers (MAG, transferrin, MOBP and MOG) in cells transfected with siMRF expressed as a percentage of control (siCont transfected cell values). The expression of OL genes was strongly inhibited in siMRF transfected cells relative to siCont transfected cells, with late-phase OL markers (transferrin, MOG and MOBP) typically being more affected than early markers (CNP 1 or Ugt8) or intermediate markers (PLP1, MBP) of differentiation. Results are averages of 3 independent experiments and expressed as mean percentages of siCont expression levels, ±SEM. D) Venn diagram showing overlap of genes induced >4-fold with differentiation and those repressed >4-fold by transfection with siMRF. The vast majority (81%) of siMRF inhibited genes were genes usually up-regulated during OL differentiation; in contrast, only 13% of genes usually induced during OL differentiation were dependent on MRF expression.

FIG. 5 is a table of OL gene expression in the absence of MRF expression. Affymetrix analysis of gene expression in cells transfected with siCont or siMRF as OPCs then cultured for 48 hours in differentiating conditions, or cultured OPCs to provide a baseline of gene expression, showing the top 50 genes displaying repressed expression in the absence of MRF. Of the top 50 genes down-regulated in the presence of siMRF, 47 were genes up-regulated 4-fold or over between OPCs and the siCont OLs. When multiple Affymetrix probe sets for the one gene was available, only the most strongly expressed is shown.

FIG. 6 shows misexpression of MRF induces OL differentiation. A-J) OPCs cultured in proliferative conditions (+PDGF, -T3) for 48 hours post transfection with pEGFP and either control (empty) vector, pSport6-MRF or pSport6-Sox10 and stained for NG2, MBP or MOG. A-C) Cells transfected with control vector (A), CMV-MRF (B) or CMV-Sox10 (C) stained for NG2. Almost all cells transfected with control vector or CMV-Sox10 (identified by co-expression of GFP) remained NG2 positive (yellow arrows). In contrast, many cells transfected with CMV-MRF down-regulated NG2 expression (green arrows), whereas untransfected cells in the same culture retained NG2 expression (red arrows). D-J) Cells transfected with control vector (D, H), pSport6-MRF (E, I) or pSport6-Sox10 (F, J) stained for MBP (D, E, F) or MOG (H, I, J). Almost all cells transfected with control vector or pSport6-Sox10 remained MBP and MOG negative (yellow arrows). In contrast, many cells transfected with pSport6-MRF were positive for MBP and MOG expression (yellow arrows). L-M) Graph displaying mean proportion of cells transfected with control vector, pSport6-MRF or pSport6-Sox10 positive for MBP (L) or MOG (M) at 48 or 120 hours post transfection. Transfection with pSport6-MRF induced a strong induction of MBP and MOG expression at both 48 and 120 hours relative to control vector transfection only, where the vast majority of cells remained MBP and MOG negative. Transfection of cells with pSport6-Sox10 resulted in a relatively modest induction of MBP and MOG at 120 hours post transfection. **P<0.01. Results are representative of 3 independent experiments.

FIG. 7 depicts electroporation of MRF in the developing chick spinal cord causes precocious MBP expression. E3 embryos were co-electroporated with pCAGGS plasmid containing MRF and EGFP and harvested at P8. A) In situ for MRF showing MRF RNA expression in the electroporated side of the spinal cord only. B) Staining for EGFP and MBP in transverse spinal cord sections. Occasional MBP+ cells (typically only 1 or 2 per section) were found on the electroporated side of the spinal cord, but not on the control side. C) Staining for EGFP and MBP in longitudinal spinal cord sections showed confinement of MBP immunoreactivity to the electroporated side. D) Higher magnification images of MBP+ cells shown in B and C.

FIG. 8 depicts analysis and generation of MRF conditional knockout mice. A) Schematic of the strategy for disruption of MRF. The wildtype MRF locus, targeting vector and locus predicted after the homologous recombination are shown. Crossing of the targeted mice with Flper mice deletes the neomycin resistance cassette resulting in mice with a loxP flanked exon 8 of MRF. Abbreviations: Ex8, exon 8; Primer 1; P2, Primer 2; P3, Primer 3; FRT, Flp recombinase site; SP, Southern Probe. B) PCR amplification of genomic DNA from a MRF wild-type, heterozygous and homozygous loxP flanked mouse. Primers 1 and 2 generated a 460 bp wildtype band, and also a 668 bp band in mice with the loxP flanked allele due to the insertion of the loxP site. Primers 1 and 3 generated a 269 bp band specific to mice with the loxP flanked allele. Primer 1, upper strand, intron 7-8. Primer 2, lower strand, exon 8. Primer 3, lower strand, plasmid insert. C) Schematic of the full-length protein coded for by the MRF gene, and the truncated post Cre-mediated excision protein lacking the DNA binding domain and subsequent C-terminal region.

FIG. 9 shows MRF conditional knockout mice displaying CNS dysmyelination. A-B) Representative images of the hippocampus, corpus callosum and overlying cortex A) and spinal cord B) of control (MRF^{wt/fl}; Olig2^{wt/Cre}) and MRF conditional knockout (MRF^{wt/fl}; Olig2^{wt/Cre}) mice stained with MBP, NeuN and GFAP at P13. C) Western blot analysis of CNP, MBP, MOG, GFAP and Neurofillament expression in the spinal cords of MRF control and conditional knockout mice at P13. D) Representative images of Fluoromyelin staining of the spinal roots and lateral white matter of the spinal cord in a control (MRF^{wt/fl}; Olig2^{wt/Cre}) and MRF conditional knockout (MRF^{wt/fl}; Olig2^{wt/Cre}) mouse at P13. E) Representative electron micrograph images of control and conditional knockout nerves at P13. Control nerves are showing a significant amount of myelination in progress. In contrast, the conditional knockout nerves never displayed myelinated axons

FIG. 10 illustrates in situ hybridization for PLP and MRF within the optic and sciatic nerves. Signal for PLP was readily detectable within both the optic and sciatic nerve (both CNS and PNS nerves), though to lower levels in the sciatic nerve, as previously reported (Puckett et al., J. Neurosci. Res. 18:511-518 (1987)). In contrast, signal for MRF was detected within the optic nerve, indicating it is expressed by OLs but not Schwann cells (expressed by CNS but not PNS glia).

FIG. 11 illustrates A-B) Phase imaging of cultured oligodendrocytes differentiated for 48 hours after being transfected with siCont (A) or siMRF (B). In both cases, the vast majority of cells take on the morphology of oligodendrocytes, but cells transfected with siMRF displayed less extensive processes. C-D) Surface staining for MOG and GalC (via the O1 antibody) on cultured oligodendrocytes differentiated for 72 hours after being transfected with siCont (C) or siMRF (D). Both siCont and siMRF transfected cells labeled readily with the O1 antibody, but only the siCont cells were positive for MOG.

FIG. 12 depicts A) RT-PCR (30 cycles) to detect MRF and MBP in cultured oligodendrocytes transfected with either a control pool of siRNA or pooled siRNA pools against MRF. A clear reduction in both MRF and MBP transcript levels were present in the siMRF transfected cells. B) RT-PCR (30 cycles) to detect MRF and MBP in cultured oligodendrocytes transfected with either a control pool of siRNA individual siRNAs against MRF. Several of the 4 independent siRNAs caused a detectable decrease in MRF and MBP levels relative to the siCont transfected cells. C) Cells from the same transfections as B) shows the percentage of viable cells expressing MOG after 4 days culture in differentiating conditions. Three of the 4 individual siRNAs against MRF caused a significant reduction in the proportion of cells expressing MOG relative to siCont transfected cells, correlating well with observed knockdown of MRF in B).

FIG. 13 illustrates MRF conditional knockouts display a loss of mature oligodendrocytes. A) Immunostaining for MBP, CC1, NG2, GFAP and Olig2 co-stained with CC1 and PDGFRα within the optic nerves of control (MRF^{wt/fl}; Olig2^{wt/Cre} and MRF^{wt/fl}; Olig2^{wt/Cre}) and MRF conditional knockout (MRF^{wt/fl}; Olig2^{wt/Cre}) mice at P13. Scale bar=50 μm. B) Quantification of the density of Olig2 immunopositive nuclei within the optic nerves. C) Quantification of the density of Olig2+/CC1+ double-immunopositive OLs within the optic nerves. D) Quantification of the density of Olig2+/PDGFRa+ double-immunopositive cells within the optic nerves. All results are expressed as means±SEM, n=4-5 per genotype. *P<0.05, **P<0.01. E) Densities of Olig2 immunopositive cells within the optic nerves of each genotype broken down into Olig2+ cells also positive for either CC1 (OLs), PDGFR1α (OPCs) or neither marker.

FIG. 14 illustrates gene expression in conditional knockout culture oligodendrocyte and spinal cords. Result of GeneChip analysis of culture OLs (differentiated for 4 days) derived from control (MRF^{wt/fl}; Olig2^{wt/cre}) and conditional knockout (CKO; MRF^{fl/fl}; Olig2^{wt/cre}) brains, and acutely isolated spinal cords taken from control (MRF^{wt/wt}; Olig2^{wt/wt}) and conditional knockout (MRF^{fl/fl}; Olig2^{wt/cre}) P13 mice. A) The 20 most down-regulated genes in cultured CKO OLs relative to control OLs are listed along with MAS 5.0 gene expression values in control and CKO OLs and spinal cord. Also shown are the fold-repression values for cultured OLs (control values/CKO values), spinal cords (control values/CKO values) and the siRNA experiments (siCont values/siMRF values). B) Representative probesets for PAN-OL lineage markers, OPC markers, early OL markers, late OL markers and their expression values in control and CKO OLs and spinal cords. Genes labeled as “A” were called absent, genes labeled “B” were considered to be expressed. “B” equals marginally expressed. Where more than one probe set was present for a given gene on the array, only the most strongly expressed probe set is shown.

FIG. 15 illustrates MRF deficient OPC/OL cultures display deficiencies in differentiative, but not proliferative, conditions A) Immunostaining of control (MRF^{wt/fl}; Olig2^{wt/cre}) and MRF conditional knockout (MRF^{fl/fl}; Oligr^{wt/cre}) cultures for NG2 and Ki67 in proliferative (+PDGF, −T3) conditions. In both cases, the vast majority of cells were maintained as NG2 and Ki67 positive progenitors (Ki67+ nuclei indicated by arrowheads). B) Immunostaining of control and MRF conditional knockout cultures for Ki67, CNP and MBP grown in differentiative (−PDGF, +T3) conditions for 4 days. In both cases, the vast majority of cells down-regulated Ki67 expression, took on the multipolar morphology characteristic of OLs (as visualized by CNP staining) and were negative for GFAP. Robust MBP staining was only seen in control (MRFwt/fl; Olig2wt/cre) cultures, however. Scale bars=100 μm. C) Schematic of transcriptional control of OL lineage specification and differentiation. Olig2 is required for OL lineage specification. Several genes, including Sox10, Nxk2.2, Yin Yang 1 and Olig1, are required for robust differentiation of OPCs into OLs. MRF is required for the maturation of immature OLs into mature OLs expression the full complement of myelin genes, with its induction possibly regulated by Yin Yang 1.

FIG. 16 illustrates CNP-Cre mediate deletion of MRF results in dysmyelinating phenotype equivalent to MRF^{fl/fl}; Olig2^{wt/cre} mice. A) Representative images of a P16 control (MRF^{fl/fl}; CNP^{wt/wt}) and conditional knockout MRF^{fl/fl}; CNP^{wt/cre} brain stained with MBP showing severe loss of MBP staining in the corpus callosum (cc) of the CNP-Cre mediated MRF conditional knockout. Some faintly MBP+, non-myelinating OLs are present in the brain of the conditional knockout (arrowheads). Scale bar=200 μm. B) Representative images of a control (MRF^{fl/fl}; CNP^{wt/wt}) and conditional knockout (MRF^{fl/fl}; CNP^{wt/cre}) P16 spinal cord stained with fluoromyelin. No CNS myelination is visible in the conditional knockout, though peripheral myelination (spinal roots; s.r.) appears unaffected.

FIG. 17 illustrates conditional knockout mice display increased apoptosis of cells within the optic nerve. A) Representative images of control nerves and a conditional knockout optic nerve at P10 stained with anti-MBP and anti-activated caspase-3. Scale bar=100 μm. B) Quantification of the density of activated caspase-3 immunopositive cells within control and conditional knockout optic nerves at P10 revealed a significant increase in the density of apoptotic cells in the conditional knockouts (**P<0.01. n=5-6/genotype). C) Higher magnification of boxed area of conditional knockout nerve from (A) showing a faintly MBP positive cell with a fragmented nucleus and activated caspase-3 staining (arrowhead).

FIG. 18 illustrates gene profiling of conditional knockouts. A) RT-PCR (30 cycles) analysis of select genes in RNA from the spinal cord or cultured OLs derived form control (MRF^{wt/fl}; Olig2^{wt/cre} and MRF^{fl/fl}; Olig2^{wt/wt}) and MRF conditional knockout (MRF^{fl/fl}; Olig2^{wt/cre}) mice showing loss of OL markers MOG and MOBP in conditional knockout samples. PCR product could not be detected from spinal cord or cultured OLs from conditional knockouts using primers recognizing the RNAsequence encoded by exon 8 of the gene, confirming deletion of this exon. In contrast, primers located outside MRF exon 8 detected MRF expression in conditional knockout cultured OLs, but not spinal cord.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention provides methods and compositions of modulating Gene Model 98 (GM98), also referred to as Myelin-gene Regulatory Factor (MRF), and/or its associated genes in promoting myelination/remyelination. For example, the expression of MRF can be inhibited in oligodendrocytes with siRNA targeting MRF, resulting in down-regulation of expression of many genes typically considered to be major components in production of myelin, such as Myelin Basic Protein (MBP), Myelin Oligodendrocyte Glycoprotein (MOG), myelin-associated oligodendrocyte basic protein (MOBP) and Proteolipid Protein (PLP), which can be modulated, directly or indirectly through MRF. Conversely, expression of MRF can be induced in oligodendrocyte progenitor cells (OPCs), for example, by expressing MRF using an expression plasmid containing MRF cDNA, thereby promoting remyelination.

Also provided herein are methods in screening for bioactive agent which modulate MRF expression and promote myelination. For example, mice in which exon 8 of the MRF gene (which encodes part of the DNA binding region) is flanked with loxP sites can be excised within cells of the oligodendrocyte lineage by crossing the mice with a mouse line expressing Cre recombinase behind the Olig2 promoter. The mice fail to develop MBP positive oligodendrocytes or CNS myelin, and typically die in their third postnatal week due to the extensive CNS dysmyelination. These mice may be used to screen for bioactive agents that delay death, decrease dysmyelination, and/or promote development of MBP positive OLs.

The methods and compositions described herein can be relevant for treating a neuropathy. A variety of CNS and PNS disorders, such as, but are not limited to, Multiple Sclerosis (MS), Progressive Multifocal Leukoencephalopathy (PML), Encephalomyelitis, Central Pontine Myelolysis (CPM), Anti-MAG Disease, Leukodystrophies: Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, Zellweger Syndrome, Guillain-Barre Syndrome (GBS), chronic inflammatory demyelinating polyneuropathy (CIDP), multifocual motor neuropathy (MMN), spinal cord injury (e.g., trauma or severing of), Alzheimer's Disease, Huntington's Disease, Amyotrophic Lateral Sclerosis, Parkinson's Disease, and optic neuritis, may be treated using the methods and compositions disclosed herein.

### General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### DEFINITIONS

As used in the specification and claims, the singular form “a”, “an” and “the” include plural references unless the context clearly dictates otherwise. For example, the term “a cell” includes a plurality of cells, including mixtures thereof.

The term “control” is an alternative subject, cell or sample used in an experiment for comparison purpose. A control can be “positive” or “negative”. For example, a control cell can be employed in assaying for differential expression of a gene product in a given cell of interest. The expression of the gene product of the control cell can be compared to that of a test cell, for example a test cell contacted with a bioactive agent. Furthermore, a “control” can also represent the same subject, cell or sample in an experiment for comparison of different time points. In the context for screening bioactive agent, a control cell can be a neural cell that has not been contacted with a test bioactive agent.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell.

The terms “contact”, “delivery” and “administration” can be used to mean an agent enters a subject, tissue or cell. The terms used throughout the disclosure herein also include grammatical variances of a particular term. For example, “delivery” includes “delivering”, “delivered”, “deliver”, etc. Various methods of delivery or administration of bioactive agents are known in the art. For example, one or more agents described herein can be delivered parenterally, orally, intraperitoneally, intravenously, intraarterially, transdermally, intramuscularly, liposomally, via local delivery by catheter or stent, subcutaneously, intraadiposally, or intrathecally.

The term “differentially expressed” as applied to nucleotide sequence or polypeptide sequence refers to over-expression or under-expression of that sequence when compared to that detected in a control. Under-expression also encompasses absence of expression of a particular sequence as evidenced by the absence of detectable expression in a test subject when compared to a control.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

A “subject,” “individual” or “patient” is used interchangeably herein, which refers to a vertebrate, preferably a mammal, more preferably a human. Mammals include, but are not limited to mice, rats, dogs, pigs, monkey (simians) humans, farm animals, sport animals, and pets. Tissues, cells and their progeny of a biological entity obtained in vivo or cultured in vitro are also encompassed.

As used herein, “treatment” or “treating,” or “ameliorating” are used interchangeably herein. These terms refers to an approach for obtaining beneficial or desired results including and preferably clinical results. For purposes of this invention, beneficial or desired clinical results include, but are not limited to, one or more of the following: shrinking the size of demyelinating lesions (in the context of demyelination disorder, for example), promoting OPC proliferation and growth or migration to lesion sites, promoting differentiation of oligodendrocytes, delaying the onset of a neuropathy, delaying the development of demyelinating disorder, decreasing symptoms resulting from a neuropathy, increasing the quality of life of those suffering from the disease, decreasing the dose of other medications required to treat the disease, enhancing the effect of another medication such as via targeting and/or internalization, delaying the progression of the disease, and/or prolonging survival of individuals. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The terms “agent”, “biologically active agent”, “bioactive agent”, “bioactive compound” or “biologically active compound” are used interchangeably and also encompass plural references in the context stated. Such compounds utilized in one or more combinatorial treatment methods of the invention described herein, include but are not limited to a biological or chemical compound such as a simple or complex organic or inorganic molecule, peptide, peptide mimetic, protein (e.g. antibody), nucleic acid molecules including DNA, RNA and analogs thereof, carbohydrate-containing molecule, phospholipids, liposome, small interfering RNA, or a polynucleotide (e.g. anti-sense).

Such agents can be agonists or antagonists of components of cell cycle pathways related to neural cell proliferation or differentiation. In some embodiments of the invention, it is envisioned that compounds having the same three dimensional structure at the binding site may be used as antagonists. Three dimensional analysis of chemical structure is used to determine the structure of active sites, including binding sites for polypeptides related to neural cell cycle.

The term “antagonist” as used herein refers to a molecule having the ability to inhibit a biological function of a target polypeptide. Accordingly, the term “antagonist” is defined in the context of the biological role of the target polypeptide. While preferred antagonists herein specifically interact with (e.g. bind to) the target, molecules that inhibit a biological activity of the target polypeptide by interacting with other members of the signal transduction pathway of which the target polypeptide is a member are also specifically included within this definition. A preferred biological activity inhibited by an antagonist is associated with increasing proliferation of OPCs, decreasing proliferation of OPCs, increasing differentiation of OLs, or increasing proliferation of astrocytes, and/or promoting remyelination. For example, an antagonist can interact directly or indirectly with a polypeptide related to neural cell cycle. Antagonists, as defined herein, without limitation, include oligonucleotide decoys, apatmers, anti-chemokine antibodies and antibody variants, peptides, peptidomimetics, non-peptide small molecules, antisense molecules, and small organic molecules.

The term “agonist” as used herein refers to a molecule having the ability to initiate or enhance a biological function of a target polypeptide. Accordingly, the term “agonist” is defined in the context of the biological role of the target polypeptide. While preferred agonists herein specifically interact with (e.g. bind to) the target, molecules that inhibit a biological activity of the target polypeptide by interacting with other members of the signal transduction pathway of which the target polypeptide is a member are also specifically included within this definition. A preferred biological activity inhibited by an agonist is associated with increasing proliferation of OPCs, decreasing proliferation of OPCs, increasing differentiation of OLs, or astrocytes thereby promoting remyelination. Antagonists, as defined herein, without limitation, include oligonucleotide decoys, apatmers, anti-chemokine antibodies and antibody variants, peptides, peptidomimetics, non-peptide small molecules, antisense molecules, and small organic molecules.

Agonists, antagonists, and other modulators of a neural cell proliferation/differentiation are expressly included within the scope of this invention. In certain embodiments, the agonists, antagonists, and other modulators are antibodies and immunoglobulin variants that bind to a polypeptide involved in modulating neural cell cycle, i.e., proliferation or differentiation. These agonistic, antagonistic modulatory compounds can be provided in linear or cyclized form, and optionally comprise at least one amino acid residue that is not commonly found in nature or at least one amide isostere. These compounds may be modified by glycosylation, phosphorylation, sulfation, lipidation or other processes.

The term “effective amount” or “therapeutically effective amount” refers to that amount of an antagonist that is sufficient to effect beneficial or desired results, including without limitation, clinical results such as shrinking the size of demyelinating lesions (in the context of demyelination disorder, for example), promoting OPC proliferation and growth, delaying the onset of a neuropathy, delaying the development of demyelinating disorder, decreasing symptoms resulting from a neuropathy, increasing the quality of life of those suffering from the disease, decreasing the dose of other medications required to treat the disease, enhancing the effect of another medication such as via targeting and/or internalization, delaying the progression of the disease, and/or prolonging survival of individuals. The therapeutically effective amount will vary depending upon the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose will vary depending on the particular antagonist chosen, the dosing regimen to be followed, whether it is administered in combination with other compounds, timing of administration, the tissue to be imaged, and the physical delivery system in which it is carried.

The term “antibody” as used herein includes all forms of antibodies such as recombinant antibodies, humanized antibodies, chimeric antibodies, single chain antibodies, humanized antibodies, fusion proteins, monoclonal antibodies etc. The invention is also applicable to antibody functional fragments that are capable of binding to a polypeptide involved in neural cell cycle (e.g., binding a transcription factor or protein involved in regulating neural cell proliferation/differentiation).

In one embodiment, comparatively low doses of an entire, naked antibody or combination of entire, naked antibodies are used. In some embodiments, antibody fragments are utilized, thus less than the complete antibody. In other embodiments, conjugates of antibodies with drugs, toxins or therapeutic radioisotopes are useful. Bispecific antibody fusion proteins which bind to the chemokine antigens can be used according to the present invention, including hybrid antibodies which bind to more than one antigen. Therefore, antibody encompasses naked antibodies and conjugated antibodies and antibody fragments, which may be monospecific or multispecific.

The terms “modulating”, “modulated” or “modulation” are used interchangeably and mean a direct or indirect change in a given context. For example, modulation of MRF expression results in altered neural cell differentiation and/or myelination. In another example, modulation can be that of a gene/gene product that itself can regulate expression of a gene involved with MRF.

The term “aptamer” as applied to bioactive agent includes DNA, RNA or peptides that are selected based on specific binding properties to a particular molecule. For example, an aptamer(s) can be selected for binding a particular gene or gene product involved in neural cell cycle, as disclosed herein, where selection is made by methods known in the art and familiar to one of skill in the art. Subsequently, said aptamer(s) can be administered to a subject to modulate or regulate an immune response. Some aptamers having affinity to a specific protein, DNA, amino acid and nucleotides have been described (e.g., K. Y. Wang, et al., Biochemistry 32:1899-1904 (1993); Pitner et al., U.S. Pat. No. 5,691,145; Gold, et al., Ann. Rev. Biochem. 64:763-797 (1995); Szostak et al., U.S. Pat. No. 5,631,146). High affinity and high specificity binding aptamers have been derived from combinatorial libraries (supra, Gold, et al.). Aptamers may have high affinities, with equilibrium dissociation constants ranging from micromolar to sub-nanomolar depending on the selection used. Aptamers may also exhibit high selectivity, for example, showing a thousand fold discrimination between 7-methylG and G (Haller and Sarnow, Proc. Natl. Acad. Sci. USA 94:8521-8526 (1997)) or between D and L-tryptophan (supra, Gold et al.).

### Regulated Expression of MRF

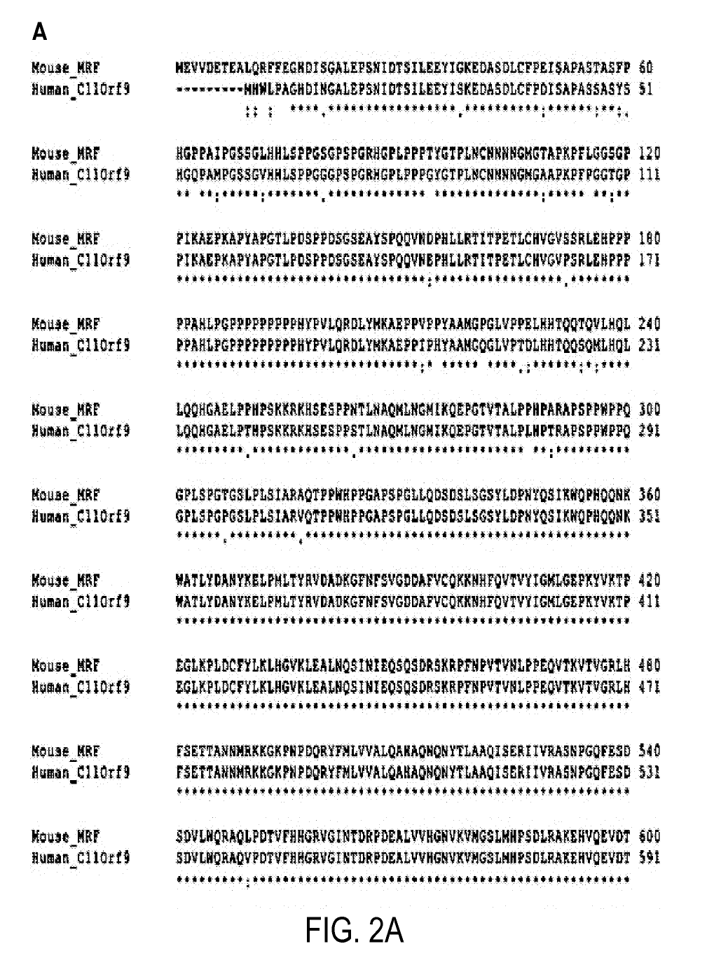

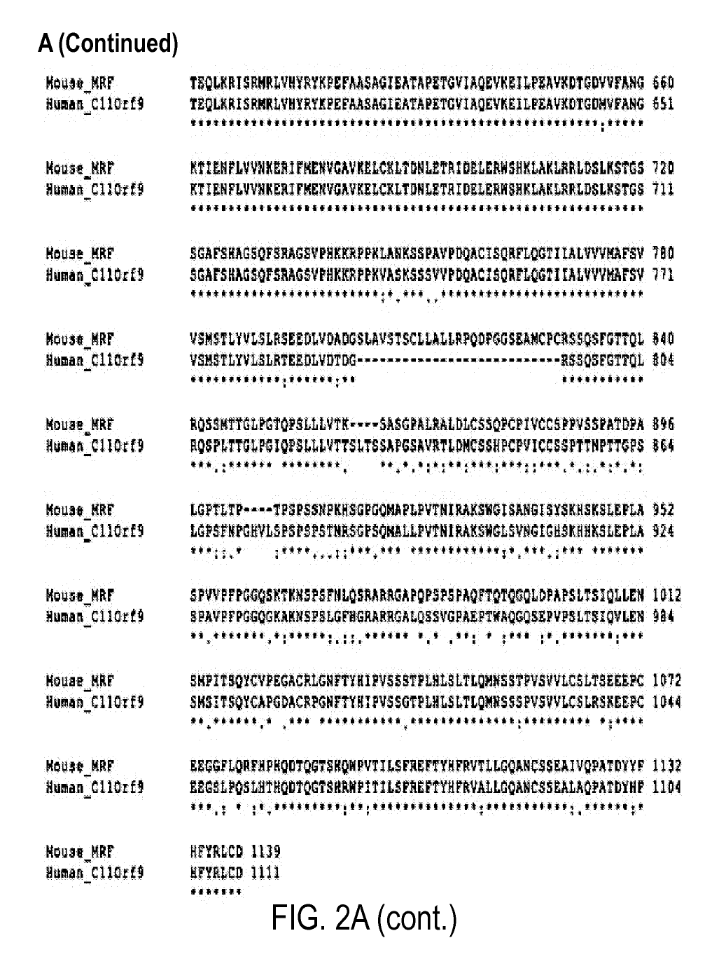

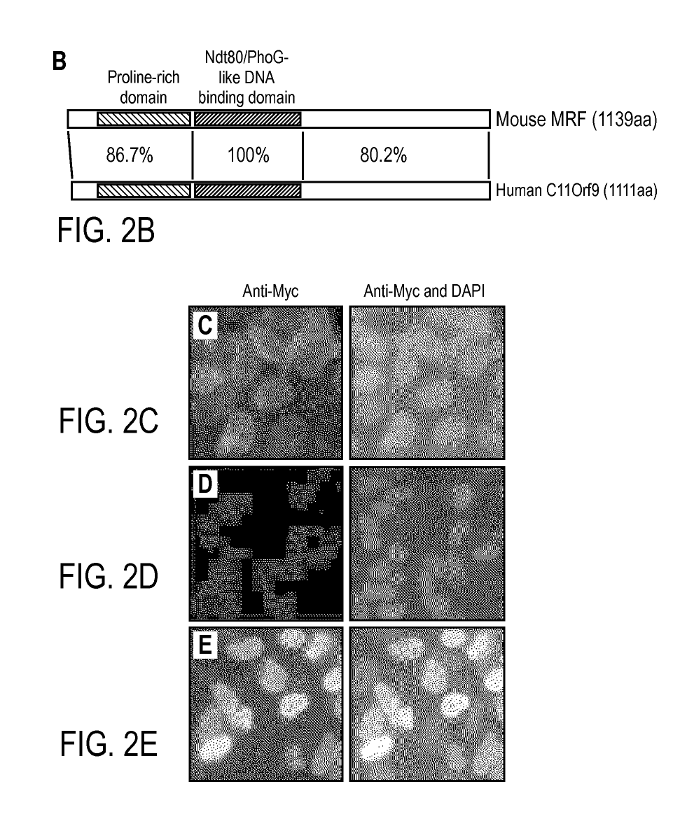

In various aspects of the present invention, a cell type, or tissue, specific expression regulatory element is operably linked to a nucleic acid sequence encoding MRF, or functional variants thereof. One or more regulatory elements may be linked to one or more nucleic sequences encoding MRF. The MRF sequence may be that of the human orthologue (C11Orf9, as shown in FIG. 2A), other orthologues, homologues, or functional variants thereof. The MRF sequence may exert a biological effect in vitro or in vivo and thus be a bioactive agent. For example, MRF can promote OL maturation and/or myelination. The regulatory elements can effect selective MRF expression, and/or provide inducible or constitutive expression of MRF. MRF expression can also be regulated or modulated by other bioactive agents.

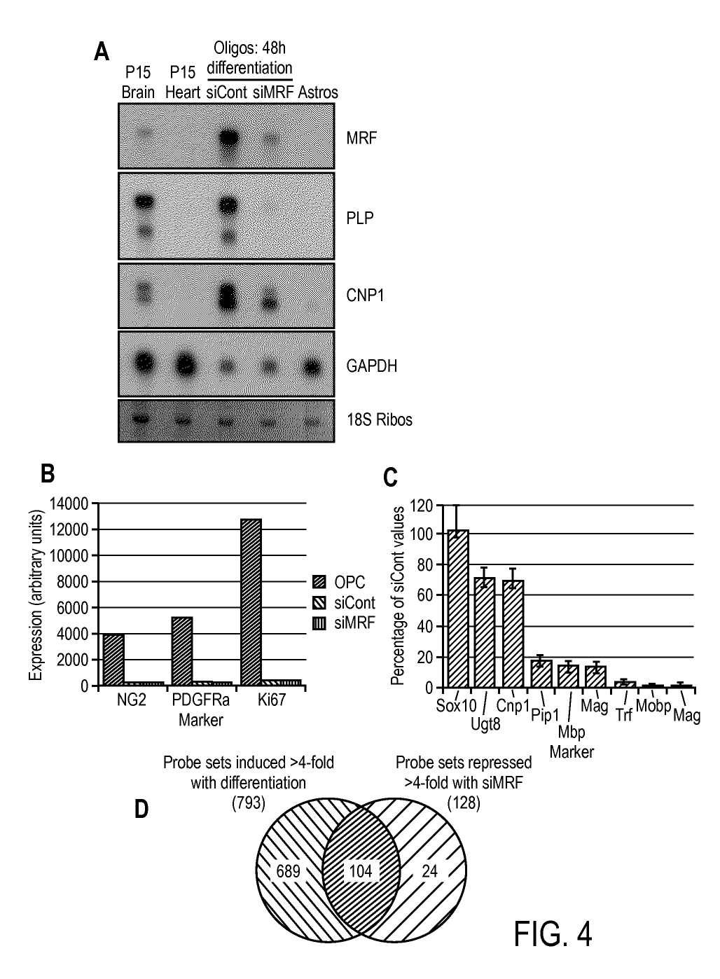

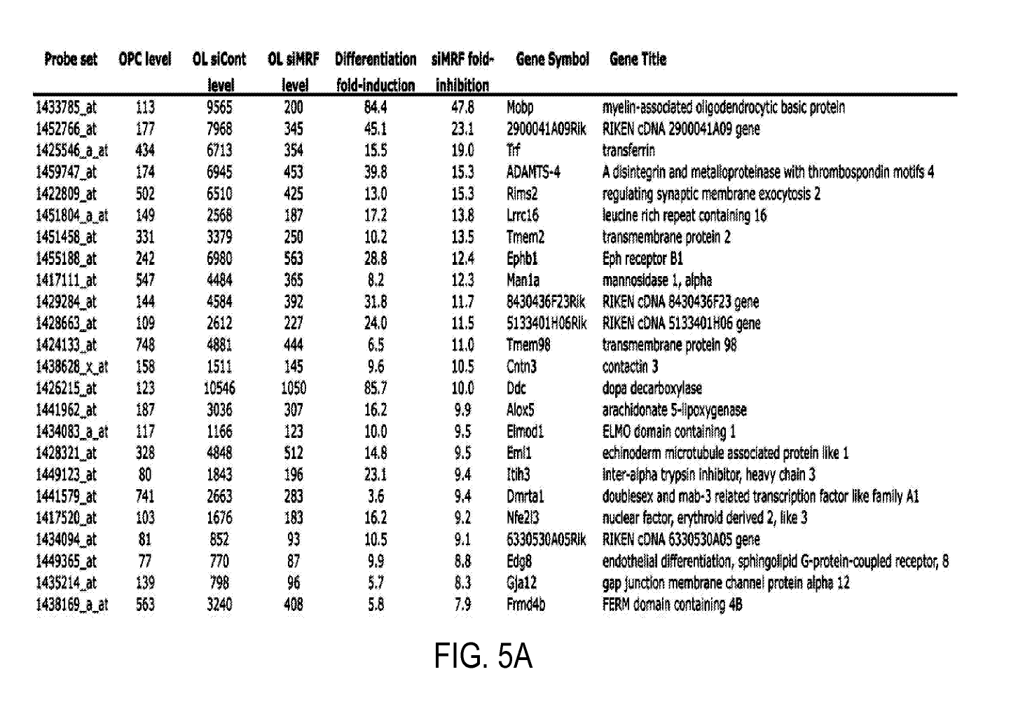

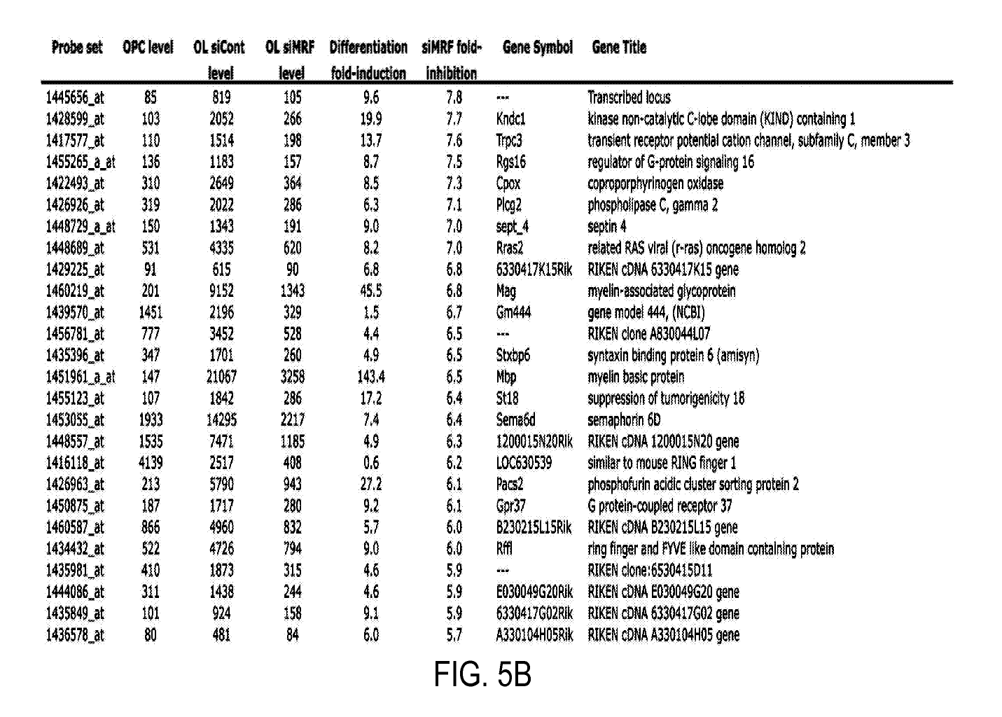

The bioactive agents utilized in the subject methods are effective in modulating the activity or expression level of MRF and its correlated genes, such as those regulated by MRF, as shown in FIG. 5, or other genes expressed during OL differentiation, such as Sox 10, Ugt8, CNP 1, Plp1, Mbp, Mag, Trf, Mobp, or Mog. Alternatively, bioactive agents effective in modulating a subset of such genes, for example, genes expressed early in OL differentiation, such as Ugt8, CNP1, Plp1, and/or Mbp; late in OL differentiation, such as Mag, Trf, Mobp, and/or Mog; or in an intermediate stage, such as Plp1, Mbp, or Mag, is also provided herein. The correlated genes may be genes specifically upregulated in mature OLs. By modulating MRF expression and/or activity, the bioactive agents affect OL maturation and myelination/remyelination. Modulation may involve augmenting or decreasing the activity or expression level of MRF. For example, an agent can be an agonist or antagonist relative to MRF, or other gene products that are implicated in MRF regulation. Non-limiting exemplary categories of such bioactive agents are peptides, antibodies, aptamers, siRNA, miRNA, EGS, antisense molecules, peptidomimetics, small molecules, pharmaceuticals, or combinations thereof.

Bioactive agents, including such as MRF, can be expressed in cells or tissues so that such agents are expressed to impart their desired function, such as promoting OL differentiation or maturation, and/or myelination. Typically, gene expression is placed under the control of certain regulatory elements, including, but not limited to, constitutive or inducible promoters, cell type specific expression regulatory elements, and enhancers. Such a gene is said to be operably linked to the regulatory elements. For example, constitutive, inducible or cell/tissue specific promoters can be incorporated into an expression vector to regulate expression of a gene that is expressed in a host cell. Therefore, depending on the promoter elements utilized, a bioactive agent can be expressed as desired so as to block, enhance or promote MRF expression or its activity. For example, an agent that promotes MRF function can be temporally expressed in cells resulting in enhanced OL differentiation, which can ultimately result in myelination/remyelination. The regulatory sequences permits ectopic expression of bioactive agents in the central nervous system or peripheral nervous system in particular cell types. For example, selective MRF modulation can be achieved in cells such as, but not limited to, neural cells, such as glial cells. Glial cells may include oligodendrocytes, microglial cells, Schwann cells or astrocytes.

Exemplary expression of regulatory sequences include regulatory sequences selected from genes including but not limited to CC1, myelin basic protein (MBP), ceramide galactosyltransferase (CGT), myelin associated glycoprotein (MAG), myelin oligodendrocyte glycoprotein (MOG), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), GFAP, AQP4, PDGFR-α, PDGF-α, RG5, pGlycoprotein, neurturin (NRTN), artemin (ARTN), persephin (PSPN), sulfatide, 2 (VEGFR2), superoxide dismutase (SOD1), tyrosine hydroxylase, neuron specific enolase, parkin gene (PARK2), parkin coregulated gene (PACRG), neuron-specific Tα1 α-tubulin (Tα1), vesicular monoamine transporter (VMAT2), and α-synuclein (SNCA), PDGFR-β, Olig1, Olig2, or proteolipid protein (PLP).

Additional examples of neural cell-specific promoters are known in the art, such as disclosed in U.S. Patent Application Publication Nos. 2003/0110524; 2003/0199022; 2006/0052327, 2006/0193841, 2006/0040386, 2006/0034767, 2006/0030541; U.S. Pat. Nos. 6,472,520, 6,245,330, 7,022,319 and 7,033,595, the relevant disclosures of which is incorporated herein by reference; See also, the website <chinook.uoregon.edu/promoters.html>; or <tiprod.cbi.pku.edu.cn:8080/index>(listing promoters of genes specific to certain cell/tissue); and Patterson et al., J. Biol. Chem. (1995)270:23111-23118.

Expression of MRF and modulators of MRF can also be temporally regulated by utilizing expression systems other than those utilizing cell type/tissue-specific promoters (e.g., where an effector molecule is administered locally). Therefore, in some embodiments, a gene encoding a cell death mediator protein can be operably linked to a controllable promoter element, such as a tet-responsive promoter. For example, where and when desired an inducible agent (e.g., tetracycline or analog thereof) can be administered to cells or a subject to induce expression of cell death mediator protein in a cell/tissue specific manner (e.g., mere tetracycline is delivered in a localized/limited manner). Such a system can provide tight control of gene expression in eucaryotic cells, by including the “off-switch” systems, in which the presence of tetracyclin inhibits expression, or the “reversible” Tet system, in which a mutant of the E. coli TetR is used, such that the presence of tetracyclin induces expression. These systems are disclosed, e.g., in Gossen and Bujard (Proc. Natl. Acad. Sci. U.S.A. (1992) 89:5547) and in U.S. Pat. Nos. 5,464,758; 5,650,298; and 5,589,362 by Bujard et al.

Additional examples of inducible promoters include but are not limited to MMTV, heat shock 70 promoter, GAL1-GAL10 promoter, metallothien inducible promoters (e.g., copper inducible ACE1; other metal ions), hormone response elements (e.g., glucocorticoid, estrogen, progestrogen), phorbol esters (TRE elements), calcium ionophore responsive element, or uncoupling protein 3, a human folate receptor, whey acidic protein, prostate specific promoter, as well as those disclosed in U.S. Pat. Nos. 6,313,373; see also, online at <biobase/de/pages/products/transpor.html> (providing a database with over 15,000 different promoter sequences classified by genes/activity); and Chen et al. Nuc. Acids. Res. 2006, 34: Database issue, D104-107.

Yet other inducible promoters include the growth hormone promoter; promoters which would be inducible by the helper virus such as adenovirus early gene promoter inducible by adenovirus E1 A protein, or the adenovirus major late promoter; herpesvirus promoter inducible by herpesvirus proteins such as VP16 or 1CP4; promoters inducible by a vaccinia or pox virus RNA polymerases; or bacteriophage promoters, such as T7, T3 and SP6, which are inducible by T7, T3, or SP6 RNA polymerase, respectively.

In other embodiments, constitutive promoters may be desirable. For example, there are many constitutive promoters suitable for use in the present invention, including the adenovirus major later promoter, the cytomegalovirus immediate early promoter, the β actin promoter, or the β globin promoter. Many others are known in the art and may be used in the present invention. In yet further embodiments, a regulatory sequence can be altered or modified to enhance expression (i.e., increase promoter strength). For example, intronic sequences comprising enhancer function can be utilized to increase promoter function. The myelin proteolipid protein (PLP) gene comprises an intronic sequence that functions as an enhancer element. This regulatory element/region ASE (antisilencer/enhancer) is situated approximately 1 kb downstream of exon 1 DNA and encompasses nearly 100 bp. See Meng et al. J Neurosci Res. 82:346-356 (2005).

Furthermore, expression of MRF, or modulators thereof, may be desired in a particular subcellular location, the nucleic acid sequence encoding MRF, or its modulator, can be operably linked to the corresponding subcellular localization sequence by recombinant DNA techniques widely practiced in the art. Exemplary subcellular localization sequences include, but are not limited to, (a) a signal sequence that directs secretion of the gene product outside of the cell; (b) a membrane anchorage domain that allows attachment of the protein to the plasma membrane or other membraneous compartment of the cell; (c) a nuclear localization sequence that mediates the translocation of the encoded protein to the nucleus; (d) an endoplasmic reticulum retention sequence (e.g. KDEL sequence) that confines the encoded protein primarily to the ER; (e) proteins can be designed to be farnesylated so as to associate the protein with cell membranes; or (f) any other sequences that play a role in differential subcellular distribution of a encoded protein product.

In other embodiments, an external guide sequence (EGS) is used to target an inhibitor of MRF (see for example, U.S. Pat. Nos. 5,728,521, 6,057,153). In one aspect, the bioactive agent of the present invention may utilize RNA interference (RNAi) as a mechanism to modulate MRF expression and/or activity. For example, RNAi may be used to target an inhibitor of MRF expression and/or activity, thereby promoting OL maturation and/or myelination. RNAi is a process of sequence-specific, post-transcriptional gene silencing initiated by double stranded RNA (dsRNA) or siRNA. RNAi is seen in a number of organisms such as Drosophila, nematodes, fungi and plants, and is believed to be involved in anti-viral defense, modulation of transposon activity, and regulation of gene expression. During RNAi, dsRNA or siRNA induces degradation of target mRNA with consequent sequence-specific inhibition of gene expression. In some embodiments, miRNA is used to target an inhibitor of MRF.

As used herein, a small interfering RNA (siRNA) is a RNA duplex of nucleotides that is targeted to a gene interest. A RNA duplex refers to the structure formed by the complementary pairing between two regions of a RNA molecule. siRNA is targeted to a gene in that the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length. The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

The bioactive agents can also be antibodies targeting one or more of the genes implicated in neural cell differentiation, for example inhibitors of MRF. Producing antibodies specific for polypeptides encoded by any of the preceding genes (or specific to active sites of the same) is known to one of skill in the art, such as disclosed in U.S. Pat. Nos. 6,491,916; 6,982,321; 5,585,097; 5,846,534; 6,966,424 and U.S. Patent Application Publication Nos. 2005/0054832; 2004/0006216; 2003/0108548, 2006/002921 and 2004/0166099, each of which is incorporated herein by reference. For example, monoclonal antibodies can be obtained by injecting mice with a composition comprising the antigen, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen that was injected, culturing the clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, for example, Coligan et al., (eds.), CURRENT PROTOCOLS IN IMMUNOLOGY, pages 2.7.1 to 2.7.12 and pages 2.9.1 to 2.9.3 (John Wiley & Sons, Inc. 1991). Also, see Baines et al., “Purification of Immunoglobulin G (IgG),” in METHODS IN MOLECULAR BIOLOGY, VOL. 10, pages 79 to 104 (The Humana Press, Inc. 1992).

Suitable amounts of well-characterized antigen for production of antibodies can be obtained using standard techniques. As an example, an antigen can be immunoprecipitated from cells using the deposited antibodies described by Tedder et al., U.S. Pat. No. 5,484,892. Alternatively, such antigens can be obtained from transfected cultured cells that overproduce the antigen of interest. Expression vectors that comprise DNA molecules encoding each of these proteins can be constructed using published nucleotide sequences. See, for example, Wilson et al., J. Exp. Med. 173:137-146 (1991); Wilson et al., J. Immunol. 150:5013-5024 (1993). As an illustration, DNA molecules encoding CD3 can be obtained by synthesizing DNA molecules using mutually priming long oligonucleotides. See, for example, Ausubel et al., (eds), CURRENT PROTOCOLS IN MOLECULAR BIOLOGY, pages 8.2.8 to 8.2.13 (1990). Also, see Wosnick et al., Gene 60:115-127 (1987); and Ausubel et al. (eds.), SHORT PROTOCOLS IN MOLECULAR BIOLOGY, 3rd Edition, pages 8-8 to 8-9 (John Wiley & Sons, Inc. 1995). Established techniques using the polymerase chain reaction provide the ability to synthesize genes as large as 1.8 kilobases in length. (Adang et al., Plant Molec. Biol. 21:1131-1145 (1993); Bambot et al., PCR Methods and Applications 2:266-271 (1993); Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263 268, (Humana Press, Inc. 1993)). In a variation, monoclonal antibody can be obtained by fusing myeloma cells with spleen cells from mice immunized with a murine pre-B cell line stably transfected with cDNA which encodes the antigen of interest. See Tedder et al., U.S. Pat. No. 5,484,892.

The bioactive agents of the present invention may also be in the form of a vector, such as a vector comprising a nucleic acid sequence encoding MRF or functional variants thereof. Vectors utilized in in vivo or in vitro methods can include derivatives of SV-40, adenovirus, retrovirus-derived DNA sequences and shuttle vectors derived from combinations of functional mammalian vectors and functional plasmids and phage DNA. Eukaryotic expression vectors are well known, e.g. such as those described by Southern and Berg, J. Mol. Appl. Genet. 1:327-341 (1982); Subramini et al., Mol. Cell. Biol. 1:854-864 (1981), Kaufmann and Sharp, 1159:601-621 (1982); Scahill et al., Proc. Natl. Acad. Sci. USA 80:4654-4659 (1983) and Urlaub and Chasin, Proc. Natl. Acad. Sci. USA 77:4216-4220 (1980), which are hereby incorporated by reference. The vector used in the methods of the present invention may be a viral vector, preferably a retroviral vector. Replication deficient adenoviruses are preferred. For example, a “single gene vector” in which the structural genes of a retrovirus are replaced by a single gene of interest, under the control of the viral regulatory sequences contained in the long terminal repeat, may be used, e.g. Moloney murine leukemia virus (MoMuIV), the Harvey murine sarcoma virus (HaMuSV), murine mammary tumor virus (MuMTV) and the murine myeloproliferative sarcoma virus (MuMPSV), and avian retroviruses such as reticuloendotheliosis virus (Rev) and Rous Sarcoma Virus (RSV), as described by Eglitis and Andersen, BioTechniques 6(7):608-614 (1988), which is hereby incorporated by reference. Expression constructs may be viral or nonviral vectors. Viral vectors that are considered part of the invention include, but are not limited to, adenovirus, adeno-associated virus, herpesvirus, retrovirus (including lentiviruses), polyoma virus, or vaccinia virus.

Recombinant retroviral vectors into which multiple genes may be introduced may also be used according to the methods of the present invention. Vectors with internal promoters containing a cDNA under the regulation of an independent promoter, e.g. SAX vector derived from N2 vector with a selectable marker (noe.sup.R) into which the cDNA for human adenosine deaminase (hADA) has been inserted with its own regulatory sequences, the early promoter from SV40 virus (SV40), may be designed and used in accordance with the methods of the present invention by methods known in the art.

Specific initiation signals can also be required for efficient translation of the nucleic sequences encoding MRF or other bioactive agents. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See e.g., Bittner et al., Methods in Enzymol. 153:516-544 (1987)).

Host cells of the present invention can be genetically modified by utilization of the foregoing nucleic acid molecules, such as those in the aforementioned vectors. Host cells can thus produce different expression levels of a gene product, such as MRF, that results in oligodendrocyte differentiation. Genetically modifying or transfecting cells either in vitro or in vivo can be conducted utilizing methods known in the art, as described in references noted herein above, and such as disclosed in U.S. Pat. No. 6,998,118, 6,670,147 or 6,465,246. Depending on the characteristics of the agent, an agent can be delivered via any of the modes of delivery known to one of skill in the art including delivery via systemic or localized delivery, delivery via plasmid vectors, viral vectors or non-viral vector systems, pharmaceutical, including liposome formulations and minicells. For example, in mammalian host cells, a number of viral-based expression systems can be utilized. In cases where an adenovirus is used as an expression vector, the nucleotide sequence of interest (e.g., encoding a therapeutic capable agent) can be ligated to an adenovirus transcription or translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the AQP1 gene product in infected hosts. (See e.g., Logan & Shenk, Proc. Natl. Acad. Sci. USA 8 1:3655-3659 (1984)). Host cells may be neural cells, such as glial cells. Neural cells may include oligodendrocytes, such as OPCs or mature OLs, as well as Schwann cells (SCs), olfactory bulb ensheathing cells, astrocytes, microglia and neural stem cells (NSCs).

Modulation of the activity or expression level of MRF can be ascertained by a variety of methods. For example, detection of a change in gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with neural cycle related genes can be performed. Typically, probes are allowed to form stable complexes with the target polynucleotides contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

As is known to one skilled in the art, hybridization can be performed under conditions of various stringencies. Suitable hybridization conditions for the practice of the present invention are such that the recognition interaction between the probe and target neural cell cycle gene is both sufficiently specific and sufficiently stable. Conditions that increase the stringency of a hybridization reaction are widely known and published in the art. (See, for example, Sambrook, et al., (1989), supra; Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical, or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

Modulation of MRF expression can also be determined by examining the corresponding gene product of MRF and/or its correlated gene products, such as those in FIG. 5, or Sox10, Ugt8, CNP1, Plp1, Mbp, Mag, Trf, Mobp, or Mog. Alternatively, expression of a subset, such as genes expressed early in OL differentiation, such as Ugt8, CNP 1, Plp1, and/or Mbp; late in OL differentiation, such as Mag, Trf, Mobp, and/or Mog; or in an intermediate stage, such as Plp1, Mbp, or Mag, may be assessed. Determining the protein level typically involves a) contacting the protein contained in a biological sample with a detection agent that specifically binds to the MRF protein, or its correlated protein; and (b) identifying any detection agent:protein complex so formed. In one aspect, the detection agent that specifically binds the protein is an antibody, such as a monoclonal antibody.

### Screening Assays

The present invention also provides a method of screening for a candidate bioactive agent effective in modulating MRF activity. The method comprises contacting a test cell with a candidate bioactive agent and assaying for a change in the expression level of MRF, or its activity, in comparison to a control cell. The candidate bioactive agent assayed in one or more methods of the present invention can also be assayed to determine if there is an overall difference in response to the bioactive agent compared at different time points, as well as compared to reference or controls.

The test cell can be a neural cell, such as a glial cell. The test cell can be, but not limited to, oligodendrocyte progenitor cells (OPC), mature OLs, Schwann cells (SCs), olfactory bulb ensheathing cells, astrocytes, microglia and neural stem cells (NSCs). The test cell can also be a stem cell or embryonic stem (ES) cell. The candidate bioactive agent can be a peptide, antibody, aptamer, siRNA, miRNA, EGS, antisense molecule, peptidomimetic, or small molecule.

The change in expression of MRF is typically indicative of a candidate bioactive agent effective in regulating differentiation of the test neural cell or test stem cell. Other changes that may also be indicative of the candidate bioactive agent's effectiveness can include changes in the expression of genes in FIG. 5, or genes specifically upregulated in mature oligodendrocytes. For example, changes in the expression of Sox10, Ugt8, CNP1, Plp1, Mbp, Mag, Trf, Mobp, or Mog. Alternatively, changes in the expression of a subset of such genes, such as genes expressed early in OL differentiation, such as Ugt8, CNP1, Plp1, and/or Mbp; late in OL differentiation, such as Mag, Trf, Mobp, and/or Mog; or in an intermediate stage, such as Plp1, Mbp, or Mag, may be assessed. The test cells can thus be utilized to screen candidate agents to determine if such agents modulate MRF, thereby promoting or inhibiting OL maturation. Bioactive agents can that are effective in increasing MRF expression or activity, can be effective in promoting OL maturation and remyelination. They may also be effective in promoting OL differentiation, such as from ES cells. Such a candidate agent can be assayed further in animal models, such as those described herein, and utilized in methods for inducing neural cell differentiation, such as in compositions and methods for treating neuropathies.

Changes in MRF expression levels can be performed by methods known in the art, including those described above. For example, changes in expression levels can be assayed by analyzing or comparing gene expression profiles of MRF from a test cell and a control cell. Changes of other genes correlated with MRF expression, such as genes listed in FIG. 5, or genes specifically upregulated in mature oligodendrocytes, such as Sox10, Ugt8, CNP1, Plp1, Mbp, Mag, Trf, Mobp, and/or Mog can be assayed. Alternatively, changes in the expression of a subset of such genes, such as genes expressed early in OL differentiation, such as Ugt8, CNP1, Plp1, and/or Mbp; late in OL differentiation, such as Mag, Trf, Mobp, and/or Mog; or in an intermediate stage, such as Plp1, Mbp, or Mag, may be assessed.

The candidate agent can be delivered in distinct temporal stages of the precursor cell cycle so as to determine if the agent affects early or late genes thus early or mature differentiated cells. For example, a candidate agent can be screened to determine if genes associated with young or mature OLs are affected, such as those induced early or late, for example, Ugt8, CNP1, Plp1, and/or Mbp in early OL differentiation, or Mag, Trf, Mobp, and/or Mog. Screening OPCs for early or late gene induction/downregulation deficiency may provide better therapeutic targeting to promote OL differentiation, by selecting agents that modulate activity of genes identified herein to be associated with early and late stage OL differentiation. Furthermore such agents can be administered to a subject to promote normal, complete maturation of OLs from different stages of undifferentiated OPCs or immature OLs. Such agents can also be utilized in reconstructing the genetic program required to produce a myelinating OL from different stages of OL differentiation.

In some aspects, the assaying step is performed in vitro. In another aspect of the method, the assaying step is performed in vivo. A variety of in vitro and in vivo methodologies are available in the art. For example, in vitro assays can be employed to promote OL differentiation in cell culture, whereas in vivo assays can be performed with animal models, as further described below. Assay of expression profiles, such as by gene chip or array technology (e.g., gene chips are readily available through multiple commercial vendors, Agilent, Affymetrix, Nanogen, etc.), immunoblot analysis, RT-PCR, and other means is well known to one of ordinary skill in the art and are also further described above.

In some aspects of the present invention, one or more candidate bioactive agents is placed in contact with a culture of cells, and before, concurrent or subsequent to such contact, one or more other bioactive agents, such as a myelin repair- or axonal protection-inducing agent is also delivered to the cells, to determine which combination of bioactive agent and myelin repair or axonal protection agent produces a synergistic effect. The one or more bioactive agents may be factors that induce stem cells, such as ES cells, to differentiate into OLs (or OPCs). For example, the factors may be transcription factors such as Sox10, Nxk2.2, Olig1, or Olig2. A synergistic effect may be observed in culture, for example, by utilizing time-lapse microscopy revealing a transition from precursor cell types to myelinating oligodendrocyte, or by assaying expression of OL specific markers, as described herein. Furthermore, progenitor cells can be transfected with a membrane-targeted form of enhanced green fluorescent protein (EGFP) to facilitate convenient fluorescence microscopy in detection of differentiated cells. Therefore, in various embodiments, cells can be cultured and/or genetically modified to express target polypeptides utilizing techniques that are known in the art, such as disclosed in U.S. Pat. Nos. 7,008,634; 6,972,195; 6,982,168; 6,962,980; 6,902,881; 6,855,504; or 6,846,625.

The cells used in screening assays may include OPCs obtained from a subject and expanded in culture from about 5, 6, 7, 8, 9 to about 14 days. The cells can be cultured for about 1, 2, 3, 4, 5, 6, 7, 8, or 9 days. Such cells can be transfected with one or more vectors during expansion in culture.

In another aspect of the present invention is a system for the screening assays. Accordingly, a representative example logic device through which data relating to the screening assays may be generated is also provided. A computer system (or digital device) to receive and store data, such as expression profiles of test cells contacted with or without a candidate bioactive agent. The computer system may also perform analysis on the data, such as comparing expression profiles between neural cells contacted with a bioactive agent, and control cells, which were not contacted with bioactive agents. The computer system may be understood as a logical apparatus that can read instructions from media and/or network port, which can optionally be connected to server having fixed media. The system typically includes CPU, disk drives, and optional input devices such as keyboard and/or mouse and optional monitor. Data communication can be achieved through the indicated communication medium to a server at a local or a remote location.

The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or review by a party. The receiving party can be but is not limited to an individual. A computer-readable medium may include a medium suitable for transmission of a result of an analysis of expression profiles resulting from neural cells contacted with a candidate bioactive agent. The medium can include a result, such as if the bioactive agent modulates the expression of MRF or other correlated genes, derived using the methods described herein.

In practicing the screening methods of the present invention, any known methods applicable to ascertain oligodendrocyte differentiation including those exemplified herein can be utilized. The candidate bioactive agents can be selected based on whether they affect promote activity (e.g., enhance expression levels of MRF) or inhibit activity (e.g., reduce expression levels or block function through binding to the target molecule, such as an inhibitor of MRF).

### Microarrays

The screening methods described herein can also be performed with the use of microarrays or gene chips that are immobilized thereon, a plurality of probes, with at least one probe corresponding to MRF. These microarrays may also be used to assess the differentiation states of oligodendrocyte-lineage cells present in several types of diseased human tissue, for example, multiple sclerosis lesions or oligodendroglioma tumor tissue. Accordingly, the present invention provides compositions comprising such microarrays.

The microarrays may include at least one probe corresponding to MRF, and one or more probes that correlate to genes regulated by MRF. The plurality of probes may correspond to MRF and at least one gene in FIG. 5. The plurality of probes may correspond to MRF and all the genes in FIG. 5. The probes can also correspond to MRF and genes specifically expressed in mature oligodendrocytes, such as Sox10, Ugt8, CNP1, Plp1, Mbp, Mag, Trf, Mobp, and/or Mog. Alternatively, the plurality of probes on the microarray can comprise MRF and a subset of genes, such as genes expressed in a discrete phase of OL differentiation, such as Ugt8, CNP1, Plp1, and/or Mbp during the early phase of OL differentiation, Mag, Trf, Mobp, and/or Mog during the late phase of OL differentiation, or genes expressed in an intermediate phase of OL differentiation, such as Plp1, Mbp, or Mag.

The probe refers to a polynucleotide used for detecting or identifying its corresponding target polynucleotide in a hybridization reaction. The term “hybridize” as applied to a polynucleotide refers to the ability of the polynucleotide to form a complex that is stabilized via hydrogen bonding between the bases of the nucleotide residues in a hybridization reaction. Different polynucleotides are said to “correspond” to each other if one is ultimately derived from another. For example, a sense strand corresponds to the anti-sense strand of the same double-stranded sequence. mRNA (also known as gene transcript) corresponds to the gene from which it is transcribed. cDNA corresponds to the RNA from which it has been produced, such as by a reverse transcription reaction, or by chemical synthesis of a DNA based upon knowledge of the RNA sequence. cDNA also corresponds to the gene that encodes the RNA. Polynucleotides may be said to correspond even when one of the pair is derived from only a portion of the other.

The arrays of the present invention may comprise control probes, positive or negative, for comparison purpose. The selection of an appropriate control probe is dependent on the sample probe initially selected and its expression pattern which is under investigation. Control probes of any kind can be localized at any position in the array or at multiple positions throughout the array to control for spatial variation, overall expression level, or non-specific binding in hybridization assays.

The polynucleotide probes embodied in this invention can be obtained by chemical synthesis, recombinant cloning, e.g. PCR, or any combination thereof. Methods of chemical polynucleotide synthesis are well known in the art and need not be described in detail herein. One of skill in the art can use the sequence data provided herein to obtain a desired polynucleotide by employing a DNA synthesizer, PCR machine, or ordering from a commercial service. Selected probes are immobilized onto predetermined regions of a solid support by any suitable techniques that effect in stable association of the probes with the surface of a solid support. By “stably associated” is meant that the polynucleotides remain localized to the predetermined region under hybridization and washing conditions. As such, the polynucleotides can be covalently associated with or non-covalently attached to the support surface. Examples of non-covalent association include binding as a result of non-specific adsorption, ionic, hydrophobic, or hydrogen bonding interactions. Covalent association involves formation of chemical bond between the polynucleotides and a functional group present on the surface of a support. The functional may be naturally occurring or introduced as a linker. Non-limiting functional groups include but are not limited to hydroxyl, amine, thiol and amide. Exemplary techniques applicable for covalent immobilization of polynucleotide probes include, but are not limited to, UV cross-linking or other light-directed chemical coupling, and mechanically directed coupling (see, e.g. U.S. Pat. Nos. 5,837,832, 5,143,854, 5,800,992, WO 92/10092, WO 93/09668, and WO 97/10365). A preferred method is to link one of the termini of a polynucleotide probe to the support surface via a single covalent bond. Such configuration permits high hybridization efficiencies as the probes have a greater degree of freedom and are available for complex interactions with complementary targets.

Typically, each array is generated by depositing a plurality of probe samples either manually or more commonly using an automated device, which spots samples onto a number of predefined regions in a serial operation. A variety of automated spotting devices are commonly employed for production of polynucleotide arrays. Such devices include piezo or ink-jet devices, automated micro-pipetters and any of those devices that are commercially available (e.g. Beckman Biomek 2000).

### Animal Models

In another aspect of the present invention, screening assays are performed in vivo. For example, a method of screening a candidate bioactive agent effective in promoting myelination can comprise administering a candidate bioactive agent to an animal and assaying for an increase in the expression level of MRF in comparison to a control animal, wherein the increase is indicative of said bioactive agent promoting myelination in the animal. The candidate bioactive agent assayed in one or more methods of the present invention can also be assayed to determine if there is an overall difference in response to the bioactive agent compared at different time points, as well as compared to reference or controls.

The animal subjects can be utilized to screen candidate bioactive agents to determine if such agents modulate MRF, thus identifying a candidate agent that either downregulates or upregulates MRF, and thereby an agent that promotes or inhibits OL maturation or differentiation and remyelination. Candidate bioactive agents useful for the subject screening methods can comprise peptide, polypeptide, peptidomimetic, antibody, antisense, aptamer, siRNA and/or small molecule. Any agents suspected to have the ability to regulate or modify MRF expression/activity, and or neural cell differentiation can be subject to the screening methods disclosed herein.

Changes of MRF and other genes correlated with MRF expression, such as genes listed in FIG. 5, or genes specifically upregulated in mature oligodendrocytes, such as Sox10, Ugt8, CNP1, Plp1, Mbp, Mag, Trf, Mobp, and/or Mog can be assayed. Alternatively, changes in the expression of a subset of such genes, such as genes expressed early in OL differentiation, such as Ugt8, CNP1, Plp1, and/or Mbp; late in OL differentiation, such as Mag, Trf, Mobp, and/or Mog; or in an intermediate stage, such as Plp1, Mbp, or Mag, may be assessed. Assaying of myelination and expression levels is well known to one of ordinary skill in the art (e.g., gene chips are readily available through multiple commercial sources) and further described herein.

The animal is typically a mammal, such as a rodent or simian species. The animal can be a mouse, rat, guinea pig, or monkey. The animal can also be a transgenic animal, such as an animal a “knock-out” or “knock-in,” with one or more desired characteristics. A “knockout” has an alteration in the target gene via the introduction of transgenic sequences that result in a decrease of function of the target gene, preferably such that target gene expression is insignificant or undetectable. A “knockin” is a transgenic animal having an alteration in a host cell genome that results in an augmented expression of a target gene, e.g., by introduction of an additional copy of the target gene, or by operatively inserting a regulatory sequence that provides for enhanced expression of an endogenous copy of the target gene. The knock-in or knock-out transgenic animals can be heterozygous or homozygous with respect to the target genes. Both knockouts and knockins can be “bigenic.” Bigenic animals have at least two host cell genes being altered.

Advances in technologies for embryo micromanipulation now permit introduction of heterologous DNA into fertilized mammalian ova. For instance, totipotent or pluripotent stem cells can be transformed by microinjection, calcium phosphate mediated precipitation, liposome fusion, retroviral infection or other means. The transformed cells are then introduced into the embryo, and the embryo will then develop into a transgenic animal. In a preferred embodiment, developing embryos are infected with a viral vector containing a desired transgene so that the transgenic animals expressing the transgene can be produced from the infected embryo. In another preferred embodiment, a desired transgene is coinjected into the pronucleus or cytoplasm of the embryo, preferably at the single cell stage, and the embryo is allowed to develop into a mature transgenic animal. These and other variant methods for generating transgenic animals are well established in the art and hence are not detailed herein. See, for example, U.S. Pat. Nos. 5,175,385 and 5,175,384.

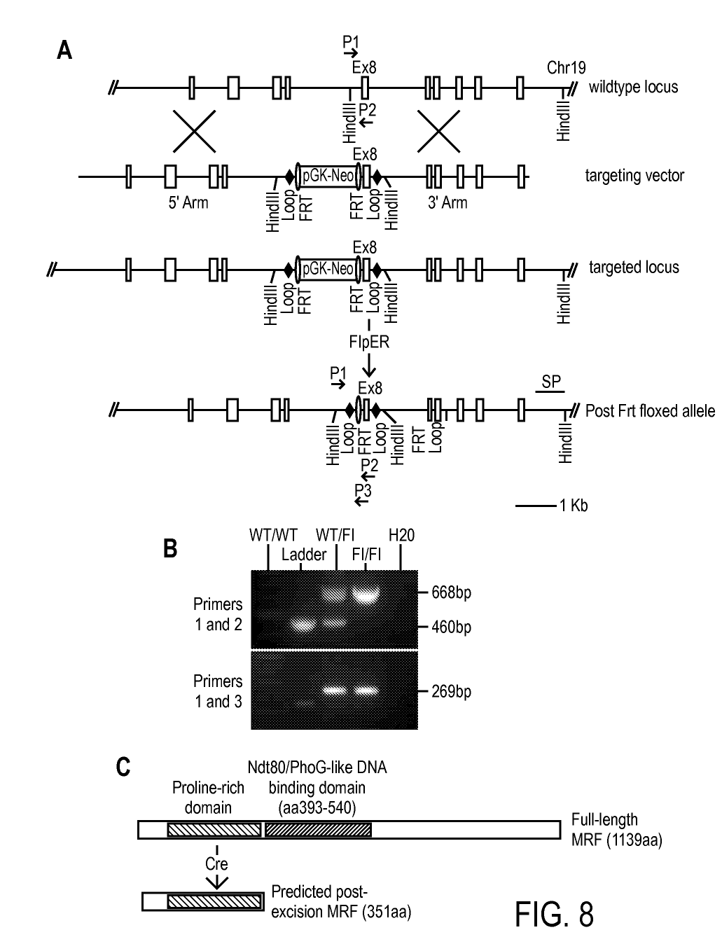

The present invention provides monogenic and bigenic animals. For example, disclosed herein are animals comprising a MRF knockin or knockout. The transgenic animal can comprise a MRF transgene, wherein the transgene is stably integrated into the animal's genome, replacing the transgenic animal's wildtype copy. Alternatively, the transgenic animal may have MRF transgene in addition to the wildtype copy of the MRF in the animal's genome. For example, the transgene may be integrated as a single copy or in concatamers, e.g., head-to-head tandems or head-to-tail tandems. The MRF transgene may have one or more mutations, for example, deletion of a particular domain or an exon. The deletion may be of an exon in the putative DNA binding domain, such as exon 8 (FIG. 8). The deletion may be constitutive or conditional. For example, the MRF transgene can be flanked by recombinase sites. The transgenic animal can also have stably integrated into its genome a sequence that encodes a recombinase, such as Cre, which recognizes the cognate recognition sequences, loxP sequences (i.e., loxP sites).

Other recombinase recognition sequences are known in the art. For example, as described, the recognition sequence for Cre recombinase is loxP which is a 34 base pair sequence comprised of two 13 base pair inverted repeats (serving as the recombinase binding sites) flanking an 8 base pair core sequence. (See Sauer, Curr. Opin. Biotech. 5:521-527 (1994)). Other examples of recognition sequences are the attB, attP, attL, and attR sequences which are recognized by the recombinase enzyme X Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins IHF, FIS, and X is. See Landy, Curr. Opin. Biotech. 3:699-707 (1993). Such sites can also be engineered according to the present invention to enhance recombination utilizing methods and products as known in the art such as disclosed in the disclosure by Hartley et al., U.S. Patent Application Publication No. 20060035269.

The Cre recombinase of the present invention may be wild type or a variant of the wild type. The Cre recombinase can be inducible in the transgenic animal (or transgenic cells). Variant Cre recombinases have broadened specificity for the site of recombination. Specifically, the variants mediate recombination between sequences other than the loxP sequence and other lox site sequences on which wild type Cre recombinase is active. In general, the disclosed Cre variants mediate efficient recombination between lox sites that wild type Cre can act on (referred to as wild type lox sites), between variant lox sites not efficiently utilized by wild type Cre (referred to as variant lox sites), and between a wild type lox site and a variant lox site. For example, the Cre variants can be used in any method or technique where Cre recombinase (or other, similar recombinases such as FLP) can be used. In addition, the Cre variants allow different alternative recombinations to be performed since the Cre variants allow much more efficient recombination between wild type lox sites and variant lox sites. Control of such alternative recombination can be used to accomplish more sophisticated sequential recombinations to achieve results not possible with wild type Cre recombinase. Variant Cre recombinases are known in the art, such as disclosed in the disclosure of U.S. Pat. No. 6,890,726. The inducibility of Cre activity may be controlled by the localization of the Cre protein. For example, the Cre protein may be a fusion of the Cre recombinase with a mutated version of the estrogen receptor, resulting in the Cre fusion, CreER^{t2}. In the absence of ligand, CreER^{t2} is cytoplasmic. However, following administration of a synthetic steroid hormone (tamoxifen), the Cre ER^{t2} protein translocates into the nucleus where it is functional (i.e., tamoxifen-inducible).

The recombinase can also be the FLP recombinase, an enzyme native to the 2 micron plasmid of Saccharomyces cerevisiae. The FLP recombinase is active at a particular 34 base pair DNA sequence, termed the FRT (FLP recombinase target) sequence. Similar to the Cre recombinase, variants, such as FlpER, that are known in the art (for example, as described in U.S. Pat. Nos. 7,371,577, 7,060,499, 6,956,146, 6,774,279), may also be used.

The transgenes of the present invention may also be selectively introduced into and activated in a particular tissue or cell type, such as cells within the central nervous system. The regulatory sequences required for such a cell-type specific activation will depend upon the particular cell type of interest, and will be apparent to those of skill in the art. For example, the nucleic acid sequence encoding the recombinase and/or the MRF transgene, can be operably linked to a cell type specific regulatory element. The regulatory element can be specific for a neural cell, such as, but not limited to, oligodendrocyte progenitor cells (OPC), mature OLs, Schwann cells (SCs), olfactory bulb ensheathing cells, astrocytes, microglia and neural stem cells (NSCs). For example, the neural cell specific regulatory element can be from a CC1, myelin basic protein (MBP), ceramide galactosyltransferase (CGT), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), GFAP, AQP4, PDGFα, RG5, pGlycoprotein, neurturin (NRTN), artemin (ARTN), persephin (PSPN), sulfatide or proteolipid protein (PLP), Olig1, or Olig2 gene. Furthermore, the cell type specific regulatory element can also be inducible or constitutive promoter.

The animal models may be used in screening assays for determining a beneficial therapeutically effective combination of bioactive agents directed to promoting remyelination, such as agents that promote immunomodulation, myelin repair/remyelinaton and/or axonal protection can be conducted utilizing animal models. For example, a transgenic animal can be modified to express or express at altered levels (i.e., up or down) an agent that promotes immunomodulation, myelin repair/remyelination or axonal protection, such as decrease or increase in expression or activity of MRF, Sox10, Nxk2.2, Olig1, and/or Olig2. Therefore, such an animal can be utilized to screen a plurality of different bioactive agents also directed to immunomodulation, myelin repair/remyelination or axonal protection, where if the transgenic animal comprises an agent directed to one end point, then the animal is administered an agent directed to a different end point(s), and vice versa, to identify a candidate combination of therapeutic agents that result in a synergistic therapeutic result for a neuropathy or related conditions described herein.

The animal model systems can be used for the development of bioactive agents that promote or are beneficial for neural remyelination. For example, a transgenic animal that is modified to express an agent resulting in an immunomodulatory, myelin repair or axonal protection phenotype, for example with increased MRF activity, can be utilized in methods of screening unknown compounds to determine (1) if a compound enhances immune tolerance, suppresses an inflammatory response, or promotes remyelination and/or (2) if a compound can result in a synergistic therapeutic effect in the animal model. Alternatively, an animal with compromised MRF activity, such as a transgenic animal with an exon 8 deletion in MRF, may be used to screen compounds that alleviates the dysmyelination in the CNS promotes the maturation of OL of the animals. Moreover, neural cells can be isolated from the transgenic animals of the invention for further study or assays conducted in a cell-based or cell culture setting, including ex vivo techniques. The model system can be utilized to assay whether a test agent imparts a detrimental effect or reduces remyelination, e.g., post demyelination insult.

The animal models may also be used to screen agents in a combinatorial manner. For example, a candidate agent can be administered with another agent, such as a second agent that effect either immunomodulation, myelin repair/remyelination or axonal protection. For example, a known bioactive agent, such as MRF, Nxk2.2, Sox10, Olig1, Olig2 or a combination thereof, can be administered to the animal, before, concurrent to, or subsequent to a candidate bioactive agent. The combinatorial effect can be determined by detecting and quantifying synergistic combinatorial treatment, such as by detecting and/or quantifying expression of cell-specific marker gene(s) and determining if and how much remyelination has occurred and if such remyelination is enhanced as compared to a control. In such an example, the control could be wild-type in which a disease model is induced, or a transgenic to which the candidate agent is not administered.

The animal models of the present invention may also be induced to undergo demyelination, and effect of the bioactive agent on remyelination may be assessed by assaying MRF expression. A number of methods for inducing demyelination in a test animal have been established. For instance, neural demyelination may be inflicted by pathogens or physical injuries, agents that induce inflammation and/or autoimmune responses in the test animal. A preferred method employs demyelination-induced agents including but not limited to IFN-γ and cuprizone (bis-cyclohexanone oxaldihydrazone). The cuprizone-induced demyelination model is described in Matsushima et al., Brain Pathol. 11:107-116 (2001). In this method, the test animals are typically fed with a diet containing cuprizone for a few weeks ranging from about 1 to about 10 weeks.

After induction of a demyelination condition by an appropriate method, the animal is allowed to recover for a sufficient amount of time to allow remyelination at or near the previously demyelinated lesions. While the amount of time required for developing remyelinated axons varies among different animals, it generally requires at least about 1 week, more often requires at least about 2 to 10 weeks, and even more often requires about 4 to about 10 weeks.

Remeylination in the animal models described herein can be ascertained by observing an increase in myelinated axons in the nervous systems (e.g., in the central or peripheral nervous system), or by detecting an increase in the levels of marker proteins of a myelinating cell, such as MRF. The same methods of detecting demyelination can be employed to determine whether remyelination has occurred. For example, demyelination/remyelination phenomena can be observed by immunohistochemical means or protein analysis as known in the art. For example, sections of the test animal's brain can be stained with antibodies that specifically recognize an oligodendrocyte marker, such as MRF. In another aspect, the expression levels of oligodendrocyte markers, such as MRF can be quantified by immunoblotting, hybridization means, and amplification procedures, and any other methods that are well-established in the art. e.g. Mukouyama et al. Proc. Natl. Acad. Sci. (2006)103:1551-1556; Zhang et al. (2003), supra; Girard et al. J. Neuroscience. (2005) 25: 7924-7933; and U.S. Pat. Nos. 6,909,031; 6,891,081; 6,903,244; 6,905,823; 6,781,029; and 6,753,456, the disclosure of each of which is herein incorporated by reference.

### Therapeutics

The compositions described herein can be used as a therapeutic. A subject with a neuropathy can be treated with a therapeutically effective amount of a bioactive agent that modulates MRF activity. A variety of neuropathies such as, but not limited to, Multiple Sclerosis (MS), Progressive Multifocal Leukoencephalopathy (PML), Encephalomyelitis, Central Pontine Myelolysis (CPM), Anti-MAG Disease, Leukodystrophies: Adrenoleukodystrophy (ALD), Alexander's Disease, Canavan Disease, Krabbe Disease, Metachromatic Leukodystrophy (MLD), Pelizaeus-Merzbacher Disease, Refsum Disease, Cockayne Syndrome, Van der Knapp Syndrome, Zellweger Syndrome, Guillain-Barre Syndrome (GBS), chronic inflammatory demyelinating polyneuropathy (CIDP), multifocual motor neuropathy (MMN), spinal cord injury (e.g., trauma or severing of), Alzheimer's Disease, Huntington's Disease, Amyotrophic Lateral Sclerosis, Parkinson's Disease, and optic neuritic, may be treated using the compositions and methods described herein.

The bioactive agents described herein can be administered to a subject to promote differentiation of oligodendrocyte progenitor cells (OPCs) into mature oligodendrocytes (OLs) by modulating MRF expression and/or activity. The neuropathy may be a demyelinating disorder, such as multiple sclerosis. Remyelination can be promoted in the subject by administering to the subject a therapeutically effective amount of a bioactive agent that modulates MRF activity.

Remyelination

Bioactive agents described herein can be administered to a subject to enhance neural cell differentiation. The bioactive agent can promote remyelination by modulating expression of MRF or its regulated genes. The bioactive agent, as described herein, can be MRF itself. Administration of such a bioactive agent can be achieved by exogenous administration of the agent itself or by providing a nucleic acid vector that encodes and expresses the agent constitutively, inducibly or in a cell specific manner, via the appropriate transcription regulatory elements described herein and known to one of ordinary skill in the art. As such the bioactive agent thus expressed can promote neural cell differentiation. Such neural cells include OLs, OPCs, SCs, NSCs, astroctyes and microglial cells.

Thus, bioactive agents that induce endogenous MRF expression can be administered to a cell/subject so as to promote neural cell differentiation and/or remyelination. In some embodiments, bioactive agents may be used to induce ES cells to differentiate into OLs (or OPCs). MRF expression can be modulated to enhance OL differentiation by administering polypeptides or nucleic acids encoding polypeptides.

Nucleic acids encoding a desired polypeptide can be transformed into target cells by homologous recombination, integration or by utilization of plasmid or viral vectors utilizing components and methods described herein and familiar to those of ordinary skill in the art. Neural cells that can be transfected include OLs, OPCs, SCs, NSCs, astocytes or microglial cells. In some embodiments, such neural cells can be transfected with more than one vector, either concurrently or at different time points. Furthermore, nucleic acids encoding any of the polypeptides disclosed herein can be operably linked to constitutive, inducible or cell-specific promoters disclosed herein, and recognized by those of ordinary skill in the art.

A bioactive agent can be administered to increase expression of a MRF resulting in neural cell differentiation, such as OL maturation to promote remyelination. The bioactive agent administered can be MRF itself. The bioactive agent administered can also be a nucleic acid vector encoding a modulator of MRF expression or encoding MRF itself. It will be evident to one of ordinary skill that nucleic acid vectors can contain constitutive, inducible or cell-specific transcription regulatory elements thus providing continuous expression of a desired bioactive agent or temporally distinct expression. For example, MRF expression can be induced in cells with doxycycline using the tetracycline repressor system. Alternatively, an expression vector can comprise a neural specific promoter, as described herein or as familiar to one of skill in the art. Therefore, in a method of treating a subject in need thereof, expression of a neural cell bioactive agent can be regulated if need be to alternate between OPC proliferation and OL differentiation to enhance remyelination.

For example, neural cells can be transfected (genetically modified) with a nucleic acid molecule that is operably linked to a constitutive, inducible or neural-cell-specific promoter and encodes MRF or another gene that modulates MRF expression/activity. Such cells can be transformed to express MRF at altered expression levels thus modulating neural cell proliferation. For example, the polypeptide can be MRF.

Growth factors or hormones can also be administered to a cell or subject to promote neural cell differentiation or proliferation. As such, growth factors and hormones may be administered concurrent to, before or subsequent to administration of any bioactive agent disclosed herein. Examples of such growth factors or hormones include thryoid hormone T3, insulin like growth factor-1, fibroblast growth factor-2, platelet-derived growth factor (PDGF), nerve growth factor, neurotrophins, neuregulins, or a combination thereof. Such growth factors or hormones can also be encoded by nucleic acid vectors that are provided concurrently, before or subsequent to any other bioactive agent disclosed herein.

Furthermore, neural cells, including, but not limited to, OPCs, Schwann cells, olfactory bulb ensheathing cells, astrocytes, microglia and neural stem cells (NSCs) can also be administered prior to, concurrent with or subsequent to administration of a bioactive agent. The one or more types of neural cells can be administered with one or more types of bioactive agents. For clarity, type means for example different types of cells (e.g., oligodendrocyte and astrocyte) or different types of bioactive agents (e.g., antibody and antisense).

Different bioactive agents may be administered, for example a first bioactive agent may be administered concurrent to, before or subsequent to administration of a second bioactive agent. More than one bioactive agent may be administered. For example, a first bioactive agent may be one that modulates MRF activity, or MRF itself. A second bioactive agent, such as one that promotes the activity of Sox10, Nxk2.2, Olig1, Olig2 or a combination thereof, may be administered concurrent to, before or subsequent to administration of an agent that modulates MRF. Alternatively, MRF may be administered concurrent to, before or subsequent to administration of Sox10, Nxk2.2, Olig1, Olig2, or a combination thereof. Administration of various bioactive agents can have a synergistic effect in promoting remyelination.

It should be understood, that the foregoing is also applicable to formulation of nucleic acid vectors that can be utilized to effect transfection of target cells. Such vectors are described herein and recognized by those of ordinary skill in the art as being capable of transfecting a target cell and expressing a desired polypeptide. In sum, such vectors can also be utilized in pharmaceutical formulations or therapeutics as described herein.

Transplantation of Remyelinating Cells

Remyelination of CNS axons has been demonstrated in various animal models. Many recent studies have since demonstrated new techniques and novel mechanisms associated with the use of cell transplantation in demyelinating disease. Human OP cells isolated from adult brains were able to myelinate naked axons when transplanted into a dysmyelinating mouse mutant. Importantly, the use of adult progenitor cells avoids ethical concerns. While OP cells are responsible for endogenous remyelination, NSCs are an alternative source of cells to promote myelin repair. NSCs are found in the adult CNS, can be expanded extensively in vitro, and can differentiate to form OLs, astrocytes, or neurons. When transplanted into rodents with relapsing or chronic forms of experimental autoimmune encephalomyelitis (EAE), NSCs have been shown to migrate to areas of CNS inflammation and demyelination and to preferentially adopt a glial cell-fate. Furthermore, attenuation of clinical disease in transplanted mice was associated with repair of demyelinating lesions and decreased axonal injury. Histological analysis confirmed that transplanted NSCs differentiated predominantly into PDGFR⁺ OP cells.

In an aspect of the present invention, the subject bioactive agents can comprise cells involved in myelin repair or remyelination of denuded axons administered to a subject, wherein the cells are modified to overexpress MRF or genes upregulated by MRF. Such cells can be cultured and transfected with an appropriate vector to express a polypeptide that leads to enhanced cell maturation, or OL differentiation. The cells can also be modified to overexpress Sox10, Nxk2.2, Olig1, or Olig2. One or more cell types can be modified to overexpress MRF, Nxk2.2, Sox10, Olig1, Olig2, or combinations thereof. For example, a cell can be modified to overexpression MRF and Sox10, Nxk2.2, Olig1, or Olig2 and administered to a subject to treat a neuropathy. Different cell types can also be administered to a subject, such as OPCs and astrocytes. In some embodiments, the myelin producing cells or progenitor cells thereof include but are not limited to fetal or adult OPCs.

The cells (“cell types”) can be oligodendrocyte progenitor cells (OPC), Schwann cells (SCs), olfactory bulb ensheathing cells, astrocytes, microglia, or neural stem cells (NSCs), which can be administered prior to, concurrent with or subsequent to administration of another bioactive agent. In some embodiments, the cells may be ES cells, such as ES cells that have been induced by bioactive agents to differentiate into OPCs or OLs. In some embodiments, such cells can be administered to an animal subject to enhance neural cell differentiation, such as OL maturation.

In one embodiment, the cells are glial cells that express the NG2 proteoglycan (NG2(+) cells), which are considered to be oligodendrocyte progenitors (OPCs) in the central nervous system (CNS), based on their ability to give rise to mature oligodendrocytes. In some embodiments, oligodendrocyte progenitor cells (OPC), Schwann cells (SC), olfactory bulb ensheathing cells, astrocytes, microglia or neural stem cells (NSC) are cultured, transformed with a vector encoding a chemokine, and expanded in vitro prior to transplantation. In other embodiments, the cells may be transfected or genetically modified in vivo to express a protein encoded the MRF gene, Sox10, Nxk2.2, Olig1, and/or Olig2.

In some embodiments, oligodendrocyte progenitor cells (OPC), Schwann cells (SCs), olfactory bulb ensheathing cells, and neural stem cells (NSCs) are transfected with one or more expression vectors, using methods known in the art or disclosed herein, so as to enable expression of one or more desired bioactive agent. Such bioactive agents can modulate MRF, Nxk2.2, Sox10, Olig1, or Olig2 expression/activity.

It will be appreciated that transplantation is conducted using methods known in the art, including invasive, surgical, minimally invasive and non-surgical procedures. Depending on the subject, target sites, and agent(s) to be the delivered, the type and number of cells can be selected as desired using methods known in the art.

### Pharmaceutical Compositions

The present invention also provides compositions for treating a neuropathy in a subject comprising the bioactive agents described herein, such as MRF. Compositions may also further comprise Sox10, Nloc2.2, Olig1, Olig2, or combinations thereof. The pharmaceutical compositions contemplated include, but are not limited to, bioactive agents that are peptides, aptamers, siRNA, miRNA, EGS, antisense molecules, nucleic acid expression vectors, antibody or antibody fragments, small molecules, or combinations thereof. Such compositions can be used in therapeutically effective amounts.

Formulations of such agents are prepared for storage by mixing such agents having the desired degree of purity with optional pharmaceutically acceptable carriers, excipients or stabilizers. (Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™, or polyethylene glycol (PEG).

In a preferred embodiment, the pharmaceutical composition that comprises the bioactive agents of the present invention is in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. T his is readily accomplished by filtration through sterile filtration membranes or other methods known in the art.

The agents may also be formulated as immunoliposomes. A liposome is a small vesicle comprising various types of lipids, phospholipids and/or surfactant that is useful for delivery of a therapeutic agent to a mammal. Liposomes containing bioactive agents are prepared by methods known in the art, such as described in Eppstein et al., Proc. Natl. Acad. Sci. USA 82:3688-3692 (1985); Hwang et al., Proc. Natl. Acad. Sci. USA 77:4030-4034 (1990); U.S. Pat. Nos. 4,485,045; 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., J. National Cancer Inst 81:1484-1488 (1989)).

### EXAMPLES

### Example 1

### Isolation, Culture and Transfection of Mouse OPCs

Mouse OPCs were isolated essentially as previously described (Cahoy et al., J Neurosci 28:264-278 (2008)). Briefly, P7 C57/B6 mouse brains were isolated, diced and enzymatically dissociated to make a suspension of single cells. These cells were sequentially panned on four BSL1 (Vector Laboratories, L-1100) coated Petri dishes for 15 minutes each to deplete microglia, then panned on an anti-PDGFRα rat monoclonal (BD Pharmingen, 558774) coated Petri dish for one hour to positively select for OPCs. Non-adherent cells were washed off with DPBS, and the adherent OPCs removed from the Petri dish with trypsin. OPCs were cultured in PDL coated T175 tissue-culture flasks at 37° C., 10% CO2 in DMEM (Invitrogen, Carlsbad, Calif.) containing 2% B-27 (Invitrogen) human transferrin (100 mg/ml), bovine serum albumin (100 mg/ml), putrescine (16 mg/ml), progesterone (60 ng/ml), sodium selenite (40 ng/ml), N-acetyl-Lcysteine (5 mg/ml), D-biotin (10 ng/ml), forskolin (4.2 mg/ml), bovine insulin (5 mg/ml) (all from Sigma), glutamine (2 mM), sodium pyruvate (1 mM), penicillin-streptomycin (100 U each) (all from Invitrogen), Trace Elements B (1×; Mediatech, Herndon, Va.), CNTF (10 ng/ml; gift from Regeneron, Tarrytown, N.J.) and PDGF-AA (10 ng/ml) and NT-3 (1 ng/ml) (both from PeproTech, Rocky Hill, N.J.). For differentiation experiments, cells were transfected as below and transferred to media as above but without PDGF-AA and with triiodothyronine (T3) (40 ng/ml; Sigma).

Transfection of OPCs: Cultures of mouse OPCs were passaged by washing the flasks once with EBSS and then treated the flasks for 5 minutes at 37° C. with 1:10 Trypsin:EDTA (Sigma) in EBSS. Cells were collected with 30% FCS/DPBS, centrifuged at 220 RCF at for 15 minutes, resuspended in growth media and centrifuged at for another 15 minutes to remove traces of trypsin. The cell pellet was resuspended to 50×10⁶ cells/ml in OPC Nucleofection solution (Amaxa). One-hundred ul (5×10⁶ cells) were added to either expression constructs (˜4 ug) or siRNAs (10 ul of 20 uM of pooled or individual siRNAs against MRF or siControl nontargeting siRNA pool, Dharmacon L-056814-00, LU-056814-00, and D001206-13, respectively) and electroporated with the Amaxa nucleofection apparatus on program 0-17. Cells were then plated out at 50,000 cells/PDL-coated coverslip in 24 well plates in differentiating media for OL marker assays, 5,000 cells/PDL-coated coverslip in 24 well plates in proliferative conditions for missexpression/differentiation assays or at 5×10⁶ cells/PDL-coated 10 cm dish in differentiating media for RNA isolation.

### Example 2

### In Ovo Electroporation of Chick Embryos

Approximately 1 ul of DNA solutions (—4 ug/ul of a 1:4 pCIG and pCAGGS-MRF mix) in TE buffer with 0.2% fast green to permit visualization were injected into the neural tube lumen of Hamburger and Hamilton stage 12 embryos. Electroporation was performed using 5×50 msec pulses at 30 volts across the embryo using an ECM830 ElectroSquare Porator (Genetronics). Embryos were harvested 4 days post electroporation and immersion fixed for 2 hours in 4% PFA before being processed for in situ hybridization or immunohistochemistry as below.

### Example 3

### Immunohistochemistry, In Situ Hybridization and TEM Microscopy

Immunohistochemistry: Ten μm cryosections from perfusion-fixed mice or cells grown on PDL-coated coverslips were fixed for 10 minutes in 4% paraformaldehyde in PBS for 10 minutes, washed 3×5 mins in PBS and then incubated for 30 minutes in blocking solution (10% fetal calf serum in PBS for surface antigens, with the addition of 0.3% triton-X for intracellular antigens). Cells were incubated overnight with primary antibodies in blocking solution (1:500 rat-anti MBP, 1:500 rabbit anti NG2; Chemicon, 1:500 anti-myc monoclonal 4A6; Upstate, 1:1,000 rabbit-anti activated caspase-3, BD Pharmingen, 1:50 mouse-anti MOG clone 8-18C5, kind gift of R. Reynolds, Imperial College, London, UK). Coverslips were washed 3×5 mins in PBS and incubated with the appropriate fluorophore conjugated secondary (Molecular Probes, diluted 1:500 in blocking solution) for 30 minutes, washed 3×5 mins in PBS and mounted in DAKI fluorescent mounting medium with DAPI nuclear counterstain. Fluoromyelin (Invitrogen) staining was performed on 10 μm cryosections according to manufacturer's instructions.

In Situ Hybridization: An in situ hybridization probe corresponding to 94 lbp from the 3′ UTR was amplified from OL cDNA using the primers GGTGGGTTTGAGTTTGGAGGTT and GGGGAAACGCTCTATGAACAGG, and subcloned into the PCR-II-Topo vector (Invitrogen). The PLP probe was a kind gift of Prof William Richardson. Antisense DIG-labeled riboprobes were synthesized using T7 polymerase and DIG RNA labeling kit (Roche) as per manufacturer's instructions. In situ hybridizations were performed essentially as described (Cahoy et al., J Neurosci 28:264-278 (2008); Schaeren-Wiemers and Gerfin-Moser, Histochemistry 100:431-440 (1993)) on 10 gm sections from P16 brains and P17 optic and sciatic nerves

TEM Microscopy: Anesthetized P13 mice were perfused with PBS followed by 2% gluteraldehyde/4% paraformaldehyde in sodium cacodylate buffer. Optic nerves were dissected out and postfixed overnight at 4° C. Following treatment with 1% osmium tetroxide and 1% uranyl acetate, nerves were embedded in epon. Sectioning and electron microscopy was performed at Stanford Microbiology and Immunology Electron Microscopy Facility.

### Example 4

### Generation of Constructs

pCS6-MRF: The coding region of MRF was amplified from cultured mouse OL cDNA using the primers CCCGGGCGCCACCATGGAGGTGGTGGACGAGAC and CTCGAGGGAGGCAGCTCAGTCACACAGG. The resulting Xma1/Xho1 linked PCR fragment was ligated into Xma1/Xho1 double digested pCMV-Sport6 (pCS6) vector (Invitrogen) and confirmed with sequencing.

pCS6-Myc-MRF: The coding region of MRF minus the start codon was amplified from cultured mouse OL cDNA using the primers CTCGAGGAGGTGGTGGACGAGACCGAAG and CTCGAGGGAGGCAGCTCAGTCACACAGG. The resulting Xma1/Xho1 linked PCR fragment was ligated into Xma1/Xho1 double digested pCS6 vector. Self-annealing oligonucleotides encoding the Myc peptide (MEQKLISEEDL) were then ligated into the Xma1 site of the resulting construct to give an N-terminal myc-MRF fusion construct.

pCAGGS-MRF: The coding region of MRF was amplified from the above pCS6-MRF with Xho1 and XbaI flanked primers. The resulting PCR product was ligated into the XbaI and Xho1 sites of pCAGGS.

### Example 5

### Transfection of HEK293 Cells

HEK 293 cells were seeded at 50-70% confluence in Dulbecco's modified Eagle medium with 10% fetal bovine serum one day before transfection. Transfections were performed using Lipofectamine 2000 (Invitrogen) as per manufacturer's instructions. Cells were analyzed by immunofluorescence 48 hours post transfection.

### Example 6

### Northern hybridization

Total RNA (5-15 μg) was run on a denaturing formaldehyde gel, transferred to Hybond-N+ (Amersham) and UV crosslinked. Membranes were then pre-hybridized for approximately 2 hrs in 100 ml hybridization solution (7% SDS, 0.5 M Na2HPO4 pH 7.2, 100 ug/ml herring sperm DNA) at 68° C. Radioactively labeled DNA probes were generated from plasmids containing approximately 1 kb of MRF, CNP, PLP or GAPDH cDNA using the Prime It II random Primer kit (Stratagene) as per manufacturer's instructions with α-³²P-dCTP (GE Healthcare). Probes were spun through Probe Quant G-50 micro columns (Amersham Biosciences) for 2 min at 400 g to eliminate unincorporated 32d-CTP and heated to 95° C. for 5 min before being added to membranes in 15 ml hybridization solution. Hybridization was allowed to occur at 68° C. overnight, washed 2×10 min in 1×SSC, 0.1% SDS at RT then 3×10 min in 0.5×SSC, 0.1% SDS at 68° C. X-ray films (Amersham) were then exposed to the membrane at −80° C. before being developed. Membranes were stripped by 3 washes in boiling 0.1×SSC, 0.1% SDS and films put down overnight to check for absence of signal prior to re-probing.

### Example 7

### Affymetrix Analysis

Total RNA was isolated from cells with the RNeasy micro kit (Qiagen, Valencia, Calif.) using Qiagen on-column DNase treatment to remove any contaminating genomic DNA. The integrity of RNA was assessed using an Agilent 2100 Bioanalyzer (Agilent Technologies) and RNA concentration was determined using a Nanoprop ND-1000 spectrophotometer (Nanoprop, Rockland, Del.).

Biotinylated cRNAs for hybridization to Affymetrix 3′-arrays were prepared from 1 ug total RNA using the Affymetrix two-cycle target labeling assay with spike in controls (Affymetrix Inc., Santa Clara, Calif., 900494). Labeled-cRNA was fragmented and hybridized to Mouse Genome 430 2.0 Arrays (3′-arrays, Affymetrix, 900495) following the manufacturer's protocols.

Raw image files were processed using Affymetrix GCOS 1.3 software to calculate individual probe cell intensity data and generate CEL data files. Using GCOS and the MAS 5.0 algorithm, intensity data was normalized per chip to a target intensity TGT value of 500 and expression data and present/absent calls for individual probe sets calculated. Gene symbols and names for data analyzed with the MAS 5.0 algorithm were from the Affymetrix Netaffx Mouse430_{—}2 annotations file (http://www.affymetrix.com/support/technical/byproduct.aff?product=moe430-20). Quality control was performed by examining raw DAT image files for anomalies, confirming each GeneChip array had a background value less than 100, monitoring that the percentage present calls was appropriate for the cell type, and inspecting the poly(A) spike in controls, housekeeping genes, and hybridization controls to confirm labeling and hybridization consistency.

### Example 8

### RT-PCR

RNA prepared as per above was subject to reverse transcription using Invitrogen Superscript III reverse transcriptase as per manufacturer's instructions. cDNA was subject to amplification for 25 or 30 cycles with gene-specific primers and run on 2% agarose gels.

### Example 9

### Generation of MRF Conditional Knockout Mice

Mice in which exon 8 of MRF was flanked by loxP sites were generated by cloning exon 8 into the SalI site of the Pez-Frt-lox-DT targeting vector. A 5 kb 5′ arm and 3 kb 3′ arm were cloned into the NotI and Xho1 sites, respectively, to enable targeting of homologous recombination into E14ES embryonic stem cells. Correctly targeted neomycin resistant clones were identified by Southern blotting of HindIII digested DNA and PCR verification of the insertion of the 5′ loxP site. Targeted cells were injected into blastocytes to generate chimeric mice, which were crossed onto C57/B6 mice to generate heterozygous mice. These mice were crossed onto the FlpER strain (Farley et al., Genesis 28-106-110 (2000)) to effect deletion of the neomycin cassette, confirmed by PCR in FlpER-negative second generation mice. Heterozygous MRF floxed mice were crossed for two generations onto Olig2-Cre mice (Schuller et al., in press) or CNP-Cre mice (Lappe-Siefke et al., Nat Genet. 33:366-374 (2003)) to obtain MRF^{fl/fl}; Olig2^{wt/cre} or MRF^{fl/fl}; CNP^{wt/cre} mice. Mice were genotyped with PCRs using a common upper primer (GGGAGGGGGCTTCAAGGAGTGT) and lower primers identifying the wild-type (CCCCCAGCATGCCGATGTACAC), and floxed (CCTTTCGCCAGGGGGATCTTG) alleles. Mice positive for Cre recombinase were identified with the primers GCTAAGTGCCTTCTCTACACCTGC and GGAAAATGCTTCTGTCCGTTTG.

### Example 10

### Quantification and Statistics

When counts were performed on cultured cells (for analysis of expression of markers etc), at least 3 coverslips per condition were counted blind, with 10 fields of vision (20× objective) counted per coverslip. Means and SEMs for each condition were calculated from the average of each coverslip and conditions compared with unpaired 2-way t-tests, using Bonferoni's correction for multiple comparisons. Experiments shown are representative of at least 2 independent experiments. For quantification of cells from tissue sections, 4-6 mice were used per genotype. Three sections were analyzed per mouse, with sections 60-100 um apart photographed (×20 objective) for analysis (with counts of cells performed in Photoshop and areas of counted regions quantified in ImageJ). In the case of quantification of activated Caspase 3 immunopositive cells the number of immunopositive cells was quantified for three longitudinal optic nerve sections 60 um apart per mouse at ×20 objective, the nerves were then photographed at low power (4× objective) and areas of the nerves determined in ImageJ. 5-6 mice were used per genotype. Means and SEMs for each genotype were calculated from the average of each mouse and genotypes compared with unpaired 2-way t-tests, using Bonferroni's correction for multiple comparisons.

### Example 11

### Identification of a GM98/MRF, an OL Specific Transcript within the CNS

An immunopanning/FACS cell purification approach was combined with gene profiling to identify genes displaying cell type specificity within the mouse CNS. Gene Model 98/Myelin-gene Regulatory Factor was identified as part of the screen in which acutely purified astrocytes, neurons, oligodendrocyte progenitors (OPCs), newly differentiated oligodendrocytes (OLs) and mature, myelinating OLs were used to generate transcriptional profiles. The Affymetrix probe set for MRF (1439506_at) displayed a similar level of enrichment in OLs over the other cell types as did quintessential OL markers such as MBP, PLP and MOG, with essentially undetectable levels of expression in neurons and astrocytes, low levels of expression in OPCs and relatively strong expression within OLs.

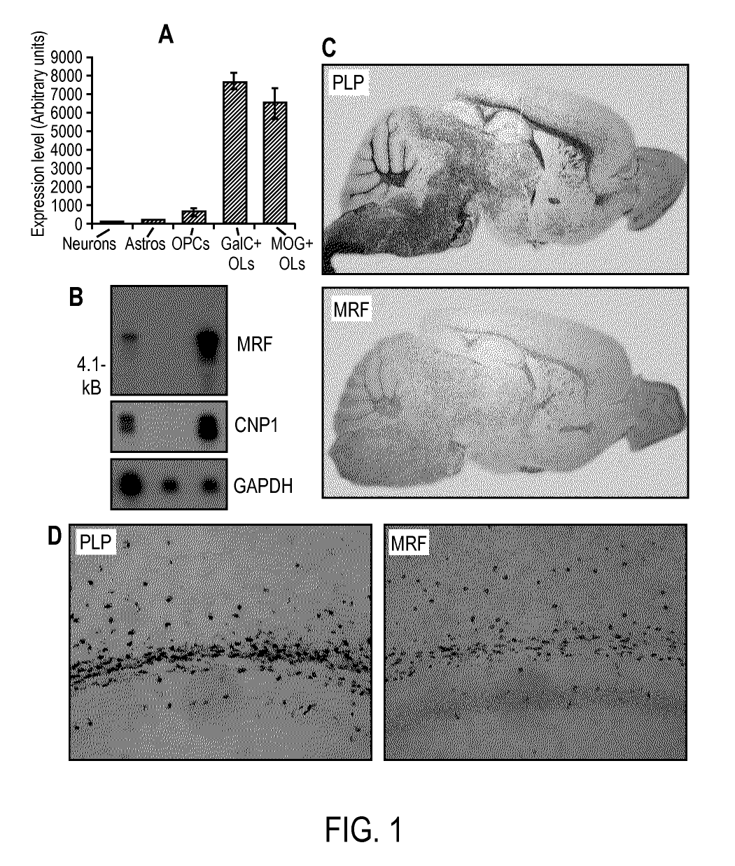

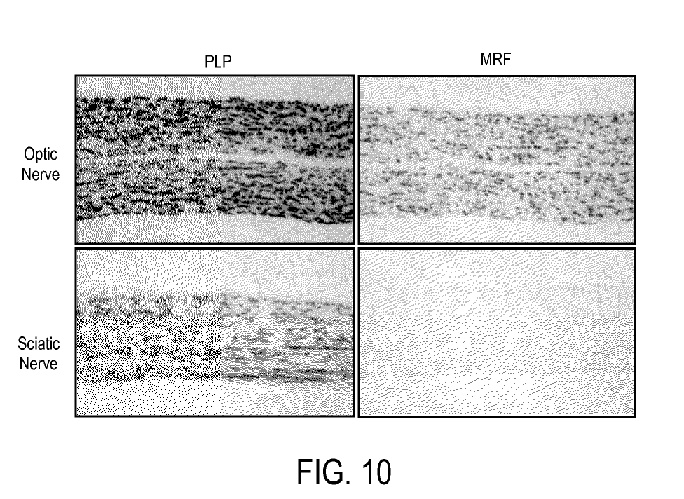

The expression of MRF was at least as high in newly differentiated OLs (GalC+, MOG−) as more mature OLs (MOG+), indicating that the gene is rapidly induced upon a transition to a postmitotic cell (FIG. 1A). Northern hybridization of RNA isolated from whole brain, heart and cultured OLs with MRF cDNA amplified from cultured OLs confirmed a clear transcript of approximately 5.5 Kb in size in brain, which was absent in samples from heart but highly enriched in cultured OLs. In situ hybridization using probes against the established OL marker Proteolipid protein (PLP) and MRF confirmed an identical expression pattern for the two genes, with expression of MRF throughout all white matter tracts in the brain (FIG. 1C), and displaying the same cellular distribution of staining within the white matter as PLP (FIG. 1D). The expression of MRF was not detected within Sciatic nerves (FIG. 10), indicating that the expression of MRF within myelinating cells is restricted to the CNS. Databases of ESTs (Unigene) indicate that MRF is expressed within other tissues, most notably the pancreas and lung, but this expression is probably considerably weaker than that seen within the CNS.

Database searching revealed that MRF is the mouse orthologue of the human gene C11Orf9. Both MRF and C11Orf9 encode a large protein which has a region of homology to the yeast transcription factor Ndt80, listed in online databases as a putative DNA binding domain (Montano et al., Proc. Natl. Acad. Sci. USA 99:14041-14046 (2002)). Sequencing of cDNA isolated from OLs indicated that the transcript for GM98/MRF encodes a protein of 1139 amino acids (FIG. 2A). This protein contains an N-terminal region containing several proline rich domains, an Ntd80-like DNA binding region and a c-terminal region containing several hydrophobic regions. Alignment of this protein with human C11Orf9 revealed an overall homology of 88.6 percent, with this homology being 100% within the DNA binding region (FIG. 2B). It has previously been suggested that C11Orf9 may be a transmembrane protein, with two hydrophobic regions within its C-terminus acting as a transmembrane helix (Stohr et al., Ctyogenet. Cell Genet. 88:211-216 (2000)). In order to establish the subcellular localization of MRF, an N-terminus Myc tagged fusion was expressed in HEK cells. This Myc-tagged protein displayed a clear nuclear expression (FIG. 2C), with a nuclear localization also seen when the tagged protein was expressed within cultured oligodendrocytes. The same nuclear subcellular localization was seen with anti-Myc antibodies directed against the N-terminal Myc tag and antibodies raised against the DNA binding region of the protein, indicating that at least these regions show a nuclear rather than membrane localization.

### Example 12

### MRF is Necessary for Myelin Gene Expression by Oligodendrocytes In Vitro

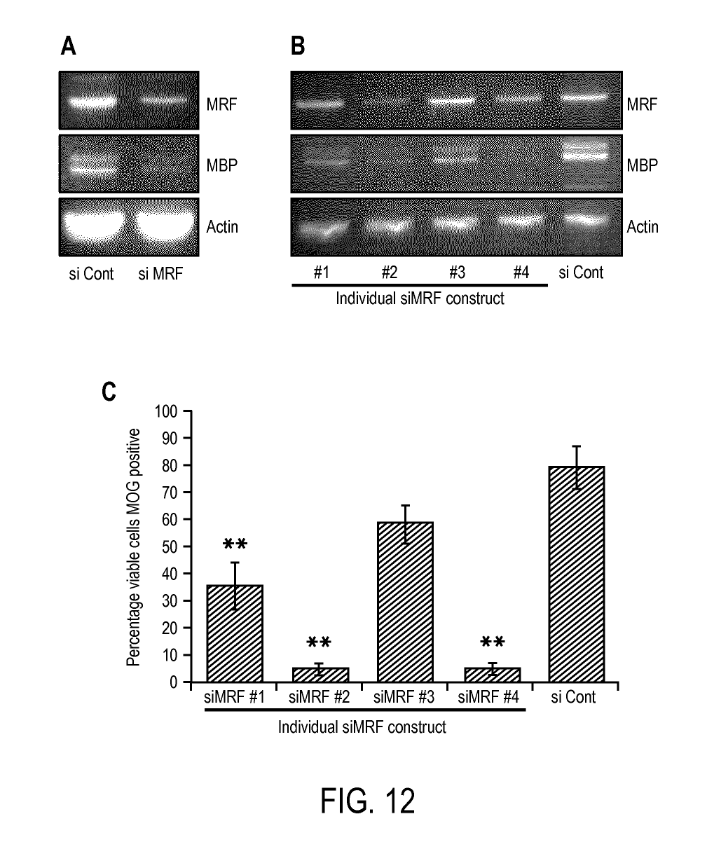

In order to establish whether MRF has a role in transcriptional regulation in OLs, the expression of MRF within OL cultures was blocked by transfecting pooled siRNAs targeting the coding region of MRF (siMRF) or non-targeting pooled siRNAs (siCont). The siMRF-transfected cells displayed a clear and consistent down-regulation of MRF mRNA relative to the siCont transfected cells as assessed by Northern blot and RT-PCR (FIG. 4A, FIG. 12), indicating that the siRNA pools were successfully reducing MRF levels. Consistent with the lack of MRF expression seen within acutely isolated OPCs, when siRNA against MRF was transfected into OPCs cultured in proliferative conditions, no effect was seen, with the vast majority of siMRF and siCont cells continuing to divide as NG2-positive progenitors.

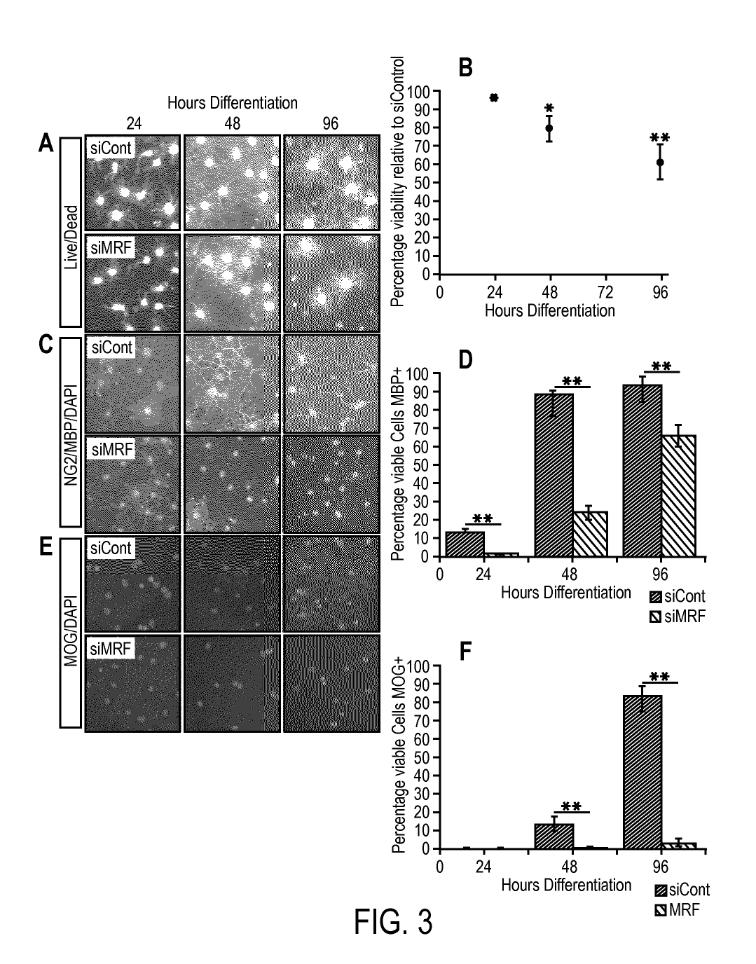

When siRNA transfected cells were transferred to differentiative conditions (−PDGF, +T3), differences began to emerge between the siCont and siMRF transfected cells. Whilst both siMRF and siCont transfected cells ceased dividing and began to extend processes within 24 hours of transfer to differentiative conditions, within 48 hours the siMRF transfected cells displayed less extensive membrane sheet deposition than the siCont transfected cells, and also displayed a modest but significantly reduced viability as assessed by a CalcienAM/EthHD1 Live/Dead assay (FIG. 3A, B). In contrast to siCont transfected cells, which were over 85% strongly MBP positive within 48 hours of induction of differentiation, siMRF transfected cells showed a clear delay and reduction in MBP expression, with only 23.6% of cells positive for MBP at 48 hours of differentiation and only weak expression in 64.4% of cells at 96 hours differentiation (FIG. 3A, C).

In spite of this, and consistent with their transition from a simple progenitor-like morphology, the siMRF transfected cells still down-regulated the expression of the OPC marker NG2 at a similar rate to the siCont transfected cells, strongly suggesting that the initial differentiation to a post-mitotic OL was unaffected by the knockdown of MRF. The expression of the late-phase OL gene MOG was found to be even more strongly inhibited in the absence of MRF, with under 5% of siMRF transfected cells expressing MOG at 96 hours differentiation, whereas 81% of siCont cells were positive for MOG at this time point (FIG. 3A, D). The reduction of cells expressing the markers MBP and MOG at 48 and 96 hours with siMRF was considerably greater than the reduction in viability (for instance, at 48 hours differentiation the siMRF transfected cells displayed only a 20.3% reduction in viability but a 62.5% reduction in the proportion of cells expressing MBP relative to siCont expressing cells), strongly suggesting that the increased cell death observed in the siMRF transfected cells was not sufficient to explain the loss of MBP and MOG expression.

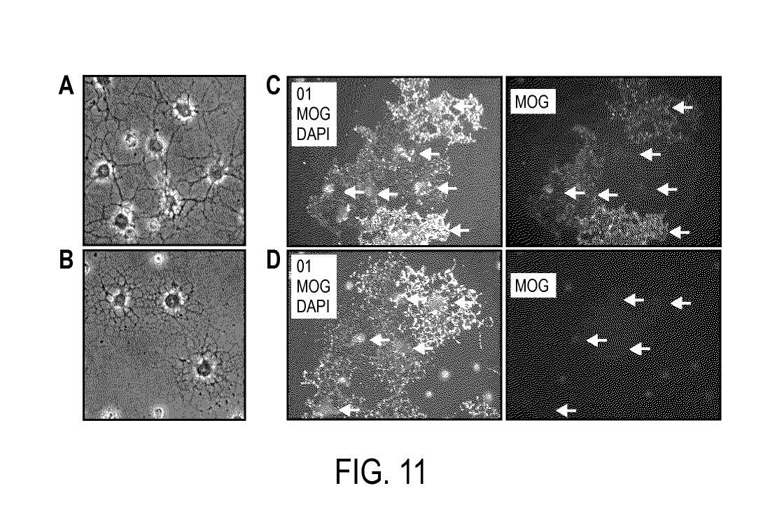

Interestingly, under phase microscopy the siMRF transfected cells almost invariably took on the general morphology of OLs, though typically with more stunted process and membrane sheet outgrowth than siCont transfected cells, and whilst MOG negative clearly expressed the OL marker GalC (FIG. 11), confirming a change in cell fate specification was not responsible for the phenotype.

### Example 13

### Identification of Genes Down-Stream of MRF

In order to further characterize the transcriptional deficits seen in differentiating OLs in the absence of MRF and in order to identify genes down-stream of MRF, RNA from OLs differentiated for 48 hours after transfection with siMRF or siCont pools, as well as from cultured OPCs to provide a baseline of gene expression prior to differentiation, was isolated. The 48 hour time point was chosen as it has been previously demonstrated that the majority of OL/myelin genes show some level of induction by this stage (Dugas et al., J. Neurosci. 26: 10967-10983 (2006)), but it was still at a time point where the siMRF transfected cells still displayed a fairly comparable level of survival relative to the siCont transfected cells, limiting the potentially {Dugas, 2006 #9} confounding effect of cell death on the results. Total RNA was used to generate labeled cRNA using a two-step linear amplification protocol with poly-A primers that amplify the 3′ end of the mRNA. This labeled cRNA was hybridized to Affymetrix Mouse 430 2.0 microarrays containing oligonucleotide probes sets complementary to 3′-end of the mRNA (3′-arrays) with 45,037 oligonucleotide probes sets representing 20,832 unique genes. Northern blot analysis was also used to confirm down regulation of MRF and myelin genes (PLP and CNP) at this time point (FIG. 4A).

Analysis of known OPC expressed genes NG2 and PDGFRα confirmed that upon the transfer of cells to differentiative conditions, these genes were down-regulated to essentially undetectable levels relative to OPCs irrespective of whether or not the expression of MRF was blocked by siRNA (FIG. 4B), confirming that MRF is not necessary for the transition from an OPC to an OL in these culture conditions. Perhaps similarly, the expression of very early OL genes such as CNP1 and Ugt8a were only slightly reduced with MRF knockdown, suggesting that in the absence of MRF, OPCs can begin to transition to OLs, and the pan-OL lineage marker Sox10 was not affected by transfection with siMRF at all. On contrast, a very clear reduction (of around 80%) in OL markers PLP1, MBP and MAG was seen with knockdown of MRF, and an almost complete reduction (>90%) of late-OL genes transferrin, MOBP and MOG relative to siCont transfected cells (FIG. 4C). Although moderately induced by transfer to differentiative conditions, due to the small percentage (>5%) of OPCs that take on a type-2 astrocyte fate in culture even in the absence of serum, possibly secondary to BMP signaling, the astroglial genes GFAP, S100b and Aquaporin 4 only showed marginal increase with MRF knockdown (147%, 255% and 153% of siCont values, respectively), confirming previous observations that the vast majority of cells remain GFAP negative in the absence of MRF expression and that diversion to the astrocytes lineage does not explain the loss of OL/myelin gene expression by these cells.

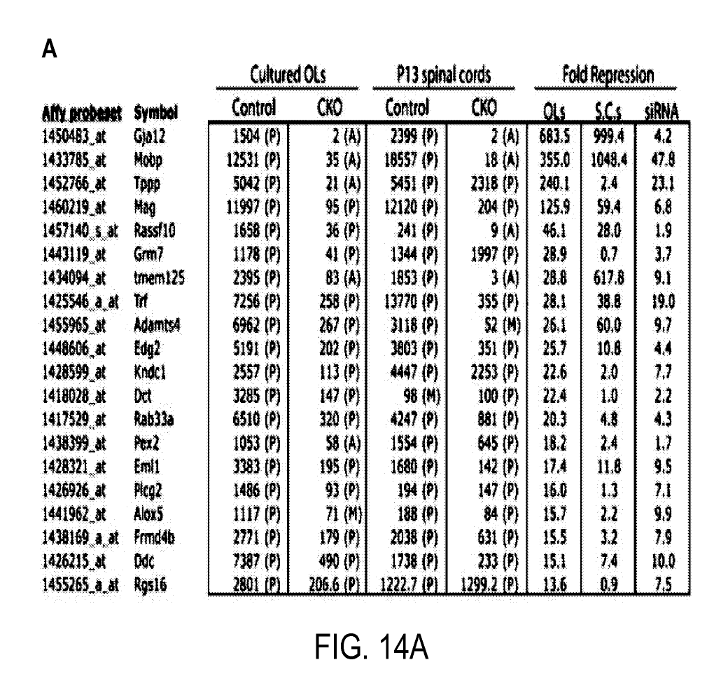

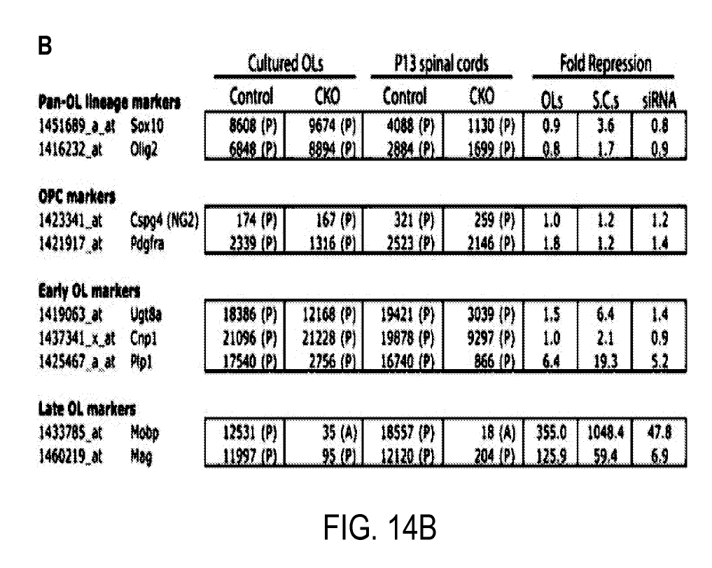

In order to identify genes downstream of MRF, genes were ordered by level of repression between siCont and siMRF. 128 probe sets representing 104 genes were >4-fold repressed with knockdown of MRF. The 50 genes showing the greatest levels of repression with MRF knockdown are shown in FIG. 5 (see also FIG. 14). Accordingly, 104 of the 128 probe sets shown to be inhibited by the siMRF were also probe sets shown to be upregulated >4 fold between OPC samples and samples of OLs differentiated for 2 days, indicating that most of the MRF dependent genes were ones usually regulated during the OPC to OL transition. Conversely, however, of the 793 probe sets induced >4 fold with differentiation, only 104 were strongly inhibited by the siMRF, suggesting that a large proportion of the genes usually regulated during OL development are independent of MRF expression (FIG. 4D).

### Example 14

### Forced MRF Expression Induces OL Differentiation In Vitro

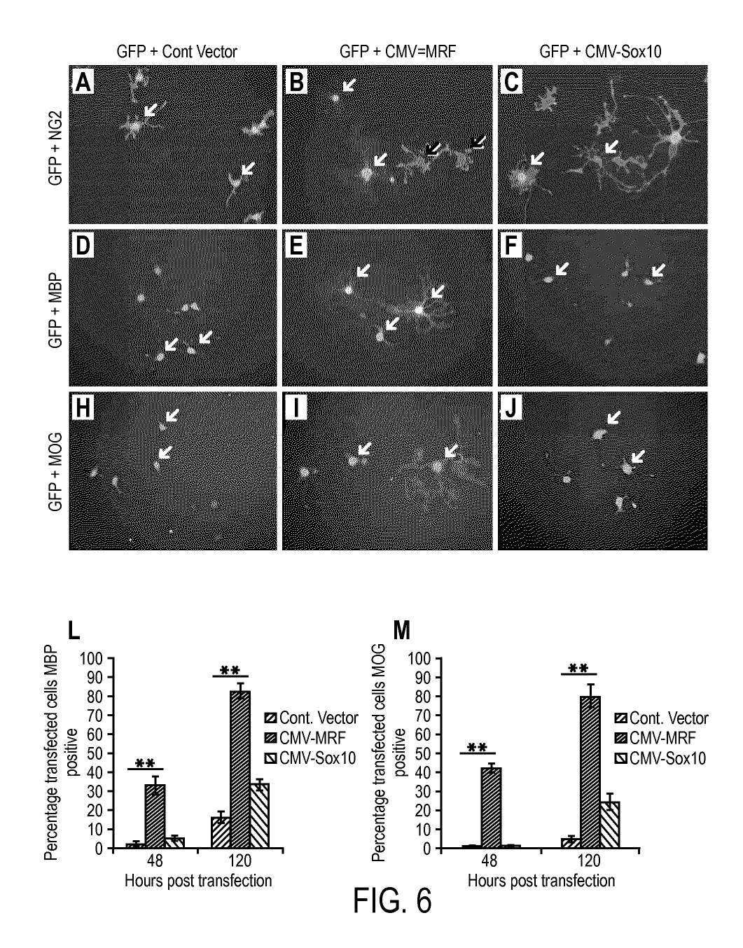

In order to assess whether MRF is sufficient to induce OL/myelin gene expression, OPCs were transfected with an MRF expression construct (pCMV-Sport6-MRF or control GFP plasmid encoding EGFP and plated into proliferative conditions (+PDGF, −T3) in which the vast majority of cells usually remain as dividing OPCs, with only low levels of spontaneous differentiation. At 2 days post transfection, the control transfected cells showed a low level of differentiation, with only 1.4 and 0.4 percent of viable cells counted MBP and MOG positive, respectively. In contrast, the cells transfected with the MRF expression construct were 32.8 and 41.0 percent positive for MBP and MOG, respectively, indicating an approximate 30-40 fold increase in the rate of differentiation (p<0.001) relative to control vector transfected cells. This induction of MBP and MOG was mirrored by a down-regulation of the OPC marker NG2 by the MRF transfected cells (FIG. 6). By 5 days post transfection, the majority of MRF transfected cells were MBP and MOG positive (81.9 and 77.2%, respectively), whereas the majority of control-transfected cells remained MBP/MOG negative OPCs (FIG. 6). The MRF misexpressing MBP and MOG positive cells almost universally displayed the general morphology of mature OLs (highly branched processes and membrane sheets), strongly suggesting that the misexpression of MRF had caused differentiation of the cells into OLs rather than the misexpression of OL/myelin genes within OPCs.

### Example 15

### Forced MRF Expression Induces MBP Expression In Vivo

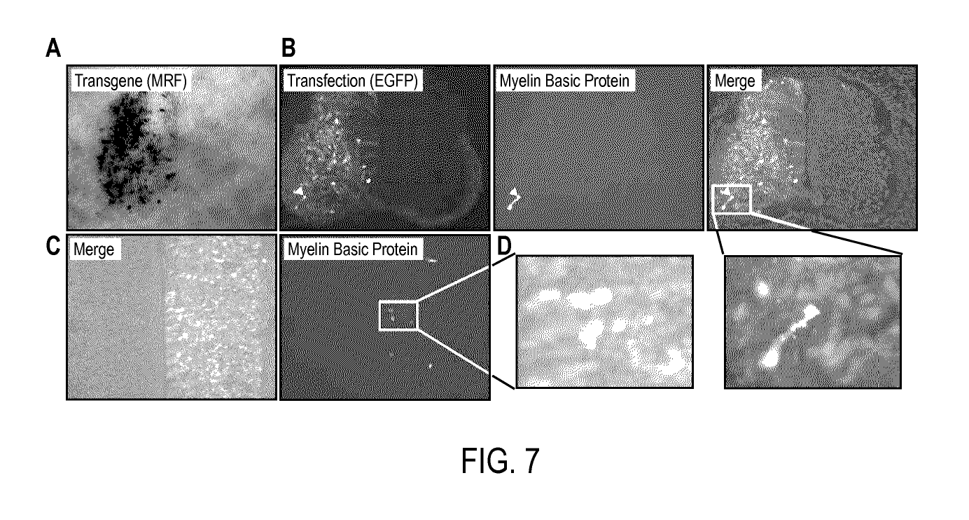

In order to assess whether the MRF expression may be sufficient to drive the expression of myelin genes in vivo, a MRF expression construct (pCAGGS-MRF) was electroporated into the chick spinal cord at E3, sacrificing the embryos at E8, a developmental time at which there is not usually detectable MBP expression or oligodendrocyte differentiation within the chick spinal cord. Within the side of the spinal cord that had been electroporated with the MRF expression construct (identifiable by expression of EGFP from a co-electroporated EGFP expression plasmid), occasional MBP positive cells could be found, though typically only 1-3 strongly MBP positive cells were found per section (FIG. 7A, B). No MBP positive cells were observed in the unelectroporated control side of the spinal cord. It should be noted, however, that the number of MBP positive cells in the electroporated side of the spinal cord was considerably less than the number of cells expressing EGFP or the MRF transgene (FIG. 7A), indicating that other positive or negative regulatory factors are likely to influence the ability of MRF to promote oligodendrocyte differentiation.

### Example 16

### MRF is Necessary for CNS Myelination in the Mouse

To assess the requirement for MRF in CNS myelination a mouse in which exon 8 of the MRF gene is flanked with loxP sites was generated (FIG. 8). Exon 8 encodes part of the DNA binding region of MRF. The deletion of this exon was predicted to result in a loss of the DNA binding region and subsequent protein due to a frame shift (FIG. 8C). Cell or tissue specific deletion of exon 8 can be effected by crossing the loxP flanked (MRF^{fl/fl}) mice with mice expressing Cre recombinase in the cell types of interest. When the MRF^{fl/fl} mice were crossed onto a mouse strain expressing Cre behind the Olig2 promoter (resulting in Cre expression in oligodendrocyte and lower motor neuron progenitors, OPCs and mature OLs), the resulting MRF conditional knockout mice (MRF^{fl/fl}, Olig2^{wt/cre}) were born at Mendelian frequencies and were not overtly distinguishable from their control littermates (MRF^{fl/fl}, Olig2^{wt/cre} and MRF^{fl/fl}, Olig2^{wt/wt}) for the first 10 days of life. Beginning at P11, however, MRF conditional knockouts were distinguishable from their littermates as they developed tremors and seizures. The conditional knockouts developed seizures over the next several days and invariably died during the third postnatal week, between P13 and P17. This phenotype is consistent with that of other mutants effecting CNS myelin.

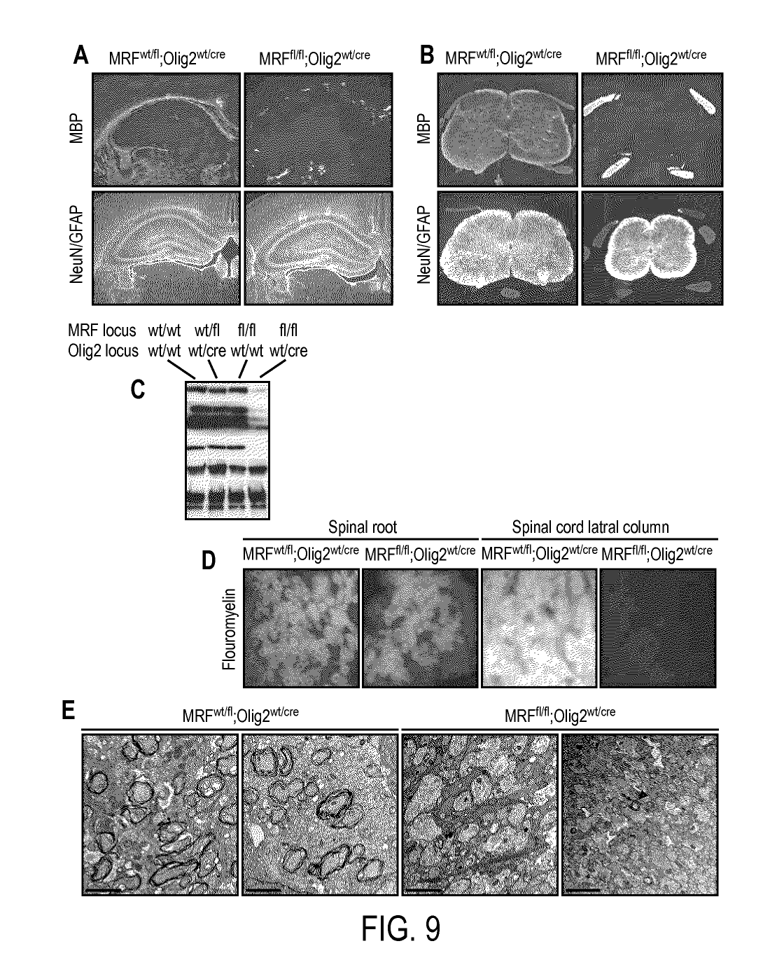

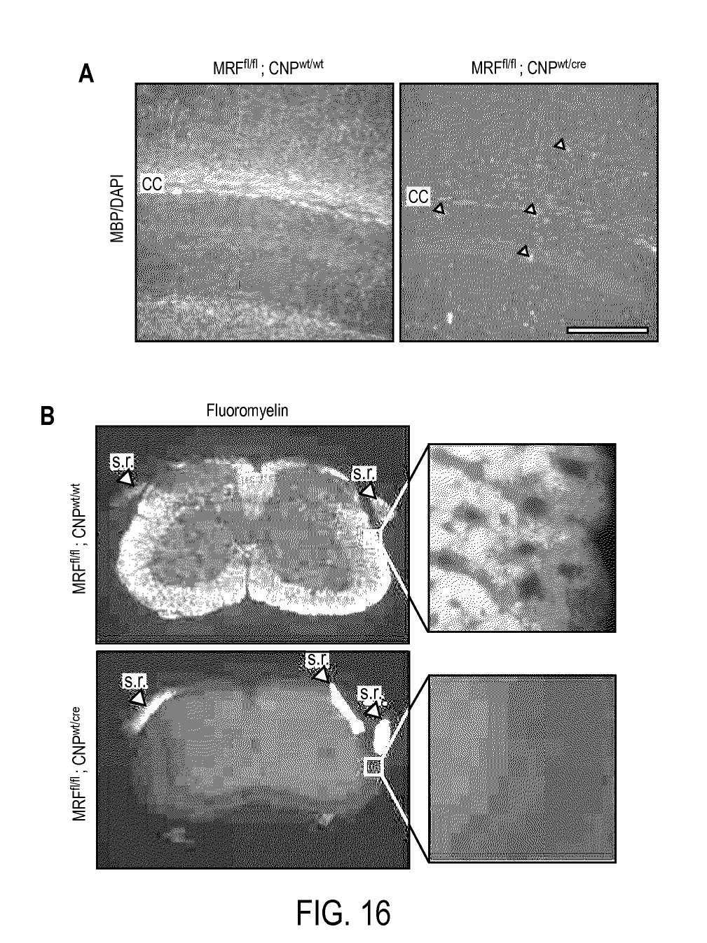

Immunohistochemical analysis of the brains and spinal cord of the conditional knockout mice at P13 indicated that NeuN and GFAP staining appeared normal, and the gross architecture of the CNS was not affected. In contrast, there was a severe loss of staining for MBP within the brain, with only occasional MBP+ oligodendrocytes seen within white matter tracts, compared to the near ubiquitous MBP staining in the white matter tracts within control mice (FIG. 9A). These infrequent remaining MBP+OLs may represent an occasional Olig2-independent cell that did not undergo Cre-mediated recombination. Within the spinal cord of conditional knockouts, this loss of MBP staining was essentially complete (FIG. 9B), although the spinal roots (myelinated by Schwann cells) still stained intensely for MBP. This loss of MBP expression was confirmed by Western blot of spinal cord lysates, which also demonstrated significantly less CNP expression and a complete loss of MOG expression (FIG. 9C). Astrocyte and neuron proteins (GFAP and neurofilament, respectively) were not affected in the conditional knockout. An essentially complete loss of remyelination was clearly evident by Fluoromyelin staining within white matter tracts, (shown for the lateral white matter of the spinal cord, FIG. 9D). In contrast, peripheral nerves were equivalently myelinated in both conditional knockouts and controls as expected, since Schwann cells express neither MRF nor Olig2. The loss of meylination was confirmed by electron microscopy in the optic nerves of conditional mice; whereas control mice were showing clear evidence of myelination in the optic nerves by P13, no myelinated nerves were observed in conditional knockout mice (FIG. 9E). An equivalent of phenotype (tremors, seizures and a loss of myelin gene expression and myelination) was seen when MRF mice were crossed with mice expressing Cre recombinase behind the CNP promoter, confirming that the conditional knockout phenotype was not dependent on the Olig2 heterozygous background caused by insertion of the Olig2-cre allele (FIG. 16). These findings confirm that MRF is necessary for the normal process of oligodendrocyte development and CNS myelination in vivo.

### Example 17

### OLS differentiate in MRF Conditional Knockout Mice but then Undergo Apoptosis as they Mature

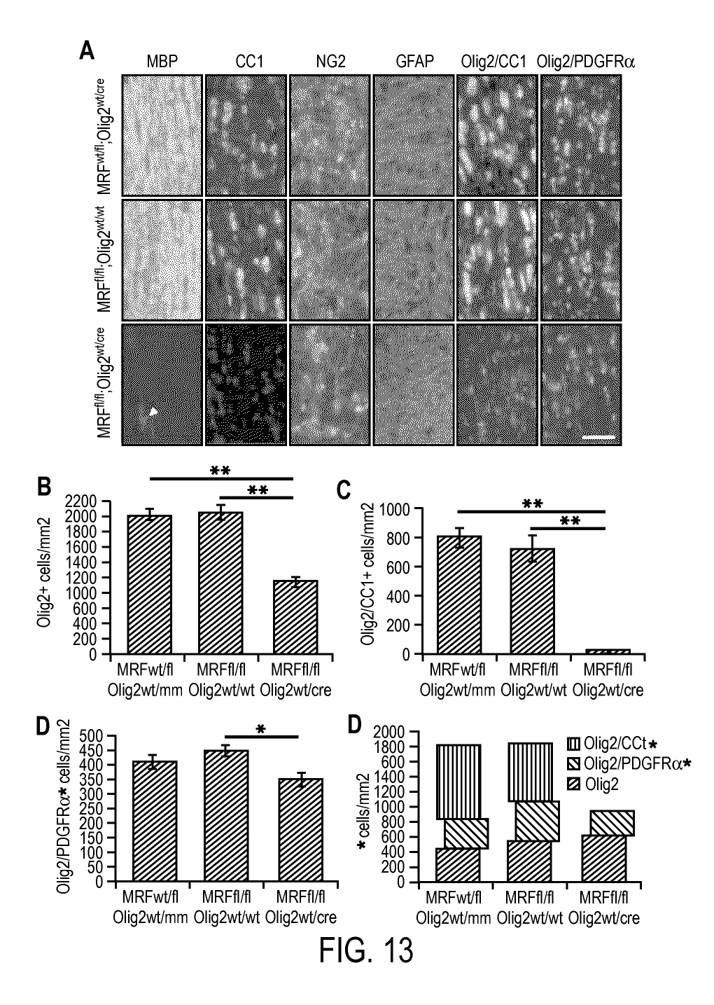

To find out whether the lack of myelin in the MRF conditional knockout mice was caused by lack of OL generation or instead by OLs that were unable to myelinate, the effect of MRF deletion on the development of each stage of the OL lineage was assessed. The densities of OPCs and OLs in P13 optic nerve sections from conditional knockout and control mice that had been immunostained for a variety of markers, including MBP, the mature OL marker CC1, OPC markers NG2 and PDGFRα and the pan-OL/astrocyte marker Olig 2 was assessed (FIG. 13A). Counts of the density of Olig2-immunopositive nuclei within the optic nerve indicated a significant reduction (of approximately 45%) of the density of positive cells in conditional knockout nerves; this reduction essentially matched the complete loss of CC1 immunopositive cells seen in conditional knockout mice (FIG. 13 B, C, E). In contrast the density of Olig2+ cells immunopositive for PDGFRα was only modestly affected in conditional knockouts (being 18.7% reduced in conditional knockouts relative to MRF^{fl/fl}; Olig2^{wt/wt} mice, t-test P<0.05, but not significantly different from MRF^{wt/fl}; Olig2^{wt/cre} controls, FIG. 13D, E), indicating the OPC stage was minimally affected in conditional knockouts, an observation supported by apparently normal NG2 staining. Similarly, the density of Olig2+ cells negative for both CC1 and PDGFRα (presumably Olig2 expression astrocytes) was similar between genotypes (FIG. 9E). Interestingly, although MBP staining in the optic nerves of conditional knockout mice was considerably reduced relative to control mice, a small number of weakly MBP+ (though CC1−) cells were nevertheless present, suggesting that at very least a small number of postmitotic OLs were generated in the conditional knockout mice. The density of these cells was very low (35±4 cells/mm²); only around 4% of the density of mature OLs as measured by CC1 staining in the control mice. The loss of MBP+ and CC1+OLs in conditional knockout mice is likely to reflect a loss of the mature OLs in addition to an inability of MRF deficient OLs to express these markers, given the loss of these markers was matched by strong reduction in the density of Olig2 positive cells, a marker not found to be downstream of MRF expression in the above siRNA experiments. Together, these data provide evidence for a severe and selective loss of the mature OL population in the MRF conditional knockouts, with the OPC population being largely unaffected.

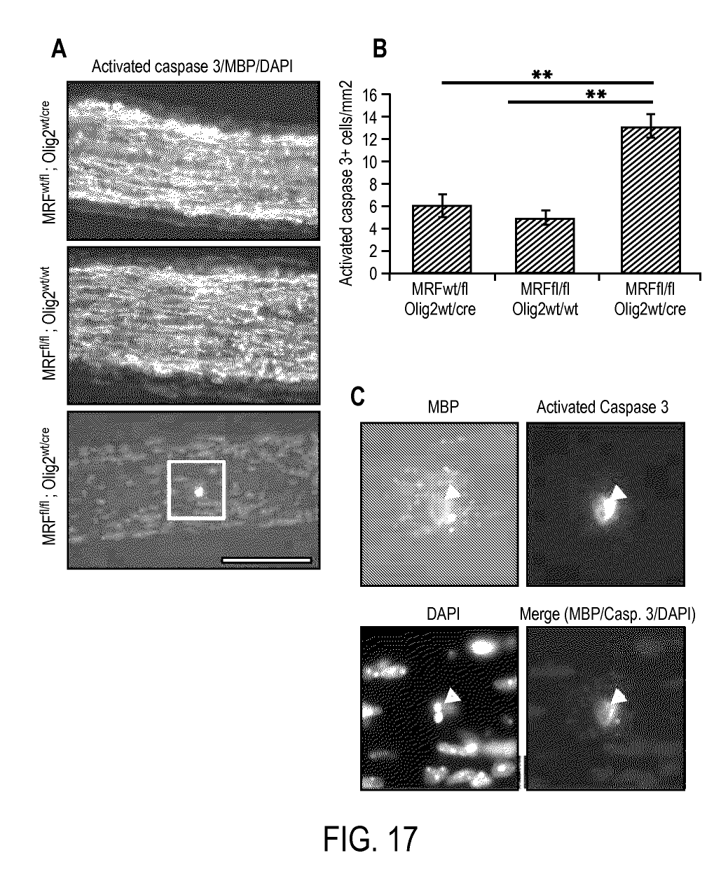

The extreme paucity of postmitotic OLs seen in conditional knockout mice may be due to either a block of differentiation of the OPCs, or, alternatively, a loss of OLs soon after their differentiation. The former possibility seemed unlikely given the above in vitro siRNA experiments indicating that OPCs lacking MRF can differentiate upon mitogen withdrawal into GC+ postmitotic cells with typical OL morphology, though they do not express myelin genes. The possibility of death of OLs was suggested by the presence of weakly MBP positive cells in conditional knockout optic nerves (FIG. 13A, arrow), some of which displayed blebbing and condensed or fragmented nuclei characteristic of apoptotic cells. In order to establish whether the observed lack of postmitotic OLs at P13 could be explained by increased apoptosis of OLs after they were generated, a cohort of P10 conditional knockout and control mice were stained with an antibody against the activated caspase-3 marker of apoptosis. Consistent with previous reports of a developmental loss of approximately half of the newly generated OLs in the optic nerve, activated caspase-3 immunopositive cells, often condensed or fragmented nuclei, were present in the optic nerves of all genotypes. Conditional knockout mice displayed a statistically significant increase of ˜2-fold in the density of these apoptotic cells in the conditional knockout mice (FIG. 17), which many of the activated caspase-3 positive cells corresponding to the weakly MBP+ cells observed in the conditional knockout nerves. These data indicate that OLs are generated in the MRF conditional knockout mice but then quickly undergo apoptosis.

### Example 18

### Dysmyelination in MRF Conditional Knockouts in Cell Autonomous and not Solely Due to Cell Death

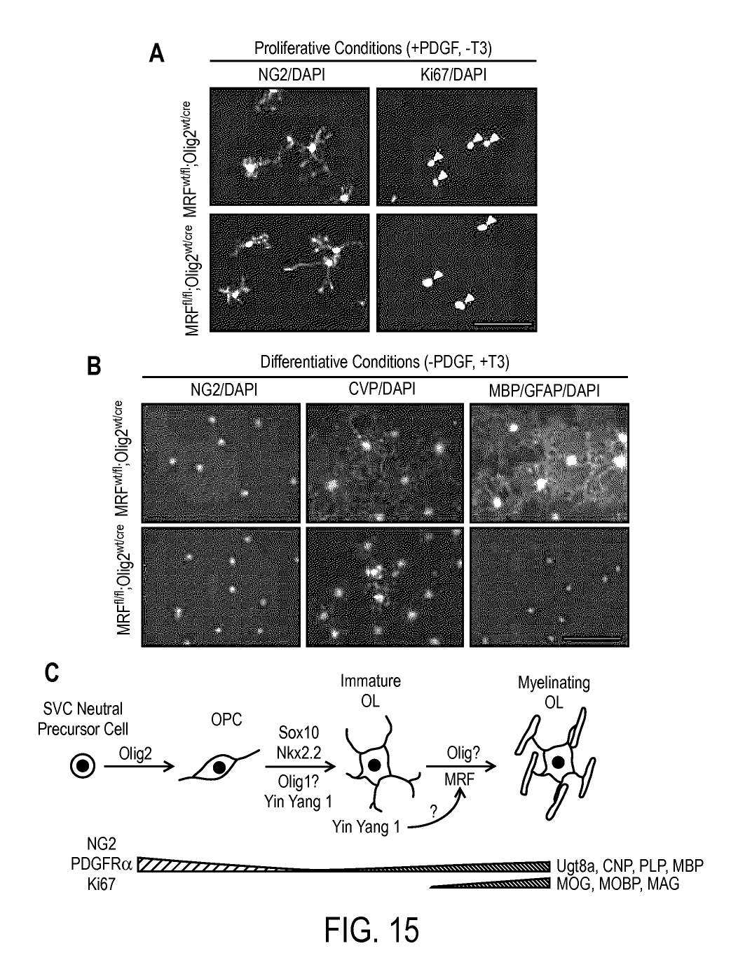

The apoptosis of OLs in the conditional knockout mice made it difficult to assess whether the dysmyelination seen in these mice was due to a loss of OLs, an inability to express myelin genes, or both. To clarify this question, highly purified OPC cultures from control and conditional mice at P7 were generated. Similar numbers of PDGFRα+ OPCs could be isolated using immunopanning from control and conditional knockout brains (˜1 million/brain). In proliferative culture conditions, the OPCs from both control and conditional knockout mice proliferated as NG2 and Ki67 positive cells, confirming MRF is not required for the OPC phase of the OL lineage (FIG. 15A).

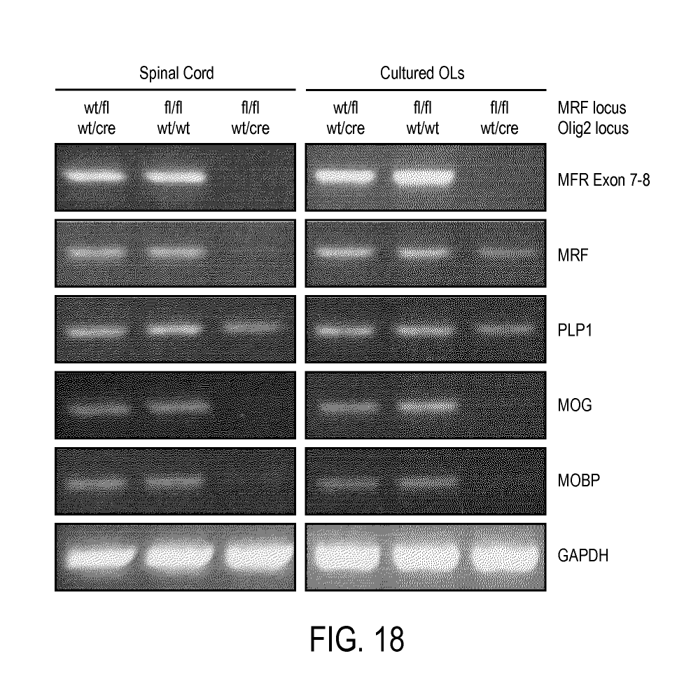

Upon withdrawal of mitogens, both control and conditional knockout cells down-regulated Ki67 and took on the morphology of OLs, staining with antibodies against CNP (FIG. 15B), and, unlike their in vivo counterparts, knockout cells exhibited almost no cell death at 4 days of differentiation. The knockout cells tended to extend considerably less extensive membrane sheets on the substrate compared to control cells, however. In spite of having excellent viability in culture, the conditional knockout cultures nevertheless failed to stain with antibodies against MBP (FIG. 15B), indicated that loss of myelin gene expression is not simply secondary to cell death. Consistent with this, when RNA was isolated from control and conditional knockout spinal cords and OL cultures and analyzed by RT-PCR and gene array, the expression of many myelin genes such as MAG and MOBP was abolished in both conditional knockout spinal cords and OL cultures, demonstrating the requirement of MRF for the expression of these genes (FIG. 14, FIG. 18). Because of the effect of MRF deficiency in inducing OL death in vivo, as expected, the loss of all MRF-dependent and—independent OL gene expression was observed in the MRF null spinal cords. The effects of MRF deficiency on OL gene expression in vitro were very similar to those observed with siMRF knockdown (FIG. 5) with most myelin genes and many OL maturation genes being strongly downregulated as expected, and including the lack of effect of MRF deficiency on several early OL genes, such as Ugt8 and Cldn11. Interestingly, several genes such as p57/cdkn1c, whose expression is normally limited to transient expression by newly-formed oligodendrocytes, were significantly upregulated in MRF-deficient OLs in vitro, indicating that MRF deficient OLs likely stall right before differentiation while they are still at an early stage of maturation before they express most myelin genes.

### Example 19

### Demyelination Inhibition by MRF Expression

A transgenic mouse with inducible MRF expression is used to show increased expression or activity of MRF in the animal inhibits demyelination. A transgenic mouse comprising inducible MRF expression is generated by crossing a PDGFRα-CreER^{T} mouse with a mouse comprising CMV-lox-stop-lox-MRF mouse, resulting in a PDGFRα-CreER^{T}/CMV-lox-stop-lox-MRF. The PDGFRα-CreER^{T}/CMV-lox-stop-lox-MRF mouse does not express the MRF from the transgene because of the upstream stop codon that is flanked by lox sites and the Cre recombinase variant, CreER^{T}, is unable to act on the floxed stop codon. However, CreER^{T} activity is induced by the administration of tamoxifen, thus when the mouse is administered tamoxifen, the stop codon is excised and MRF is expressed.

Two groups of PDGFRα-CreER^{T}/CMV-lox-stop-lox-MRF are used in this experiment, wherein one group, the test group, is administered tamoxifen. The second group, the control group, is not administered tamoxifen. Both groups are fed cuprizone to induce demyelination. Both groups are analyzed for demyelination, such as by immunohistochemistry of myelin specific genes, detection of gene expression, of myelin specific genes, or by electron micrography of axons.

The test group of mice that are administered tamoxifen exhibits a lesser degree or extent of demyelination as compared to the control group.

### Example 20

### Remyelination Promotion by MRF Expression

Two groups of PDGFRα-CreER^{T}/CMV-lox-stop-lox-MRF are used in this experiment to demonstrate promotion of remyelination by increased MRF expression or activity. Both groups are fed cuprizone to induce demyelination. One group, the test group, is administered tamoxifen, whereas the second group, the control group, is not administered tamoxifen. Both groups are analyzed for remyelination, such as by immunohistochemistry, or detection of gene expression, of myelin specific genes, or by electron micrography of axons. The test group of mice that are administered tamoxifen are able to remyelinate more quickly or more robustly than the control mice.

### Example 21

### In Vitro Screening of Bioactive Agents

A glial cell line is used to screen for bioactive agents that promote remyelination or inhibit demyelination. OPCs from mice are obtained and treated with a candidate bioactive agent. The OPCs are analyzed for MRF expression and compared to OPCs not administered the candidate bioactive agent. If the OPCs adminstered have higher levels of MRF expression as compared to the OPCs not administered the candidate bioactive agent, the candidate bioactive agent is selected for further analysis, such as for testing in an animal model as described in Example 22.

The cells can also be analyzed by immunohistochemistry, or detection of gene expression, of myelin specific genes. If the cells administered the candidate bioactive agent show increased expression of myelin specific genes or myelinated axons as compared to cells not administered the candidate bioactive agent, the candidate bioactive agent is selected for further development as a therapeutic agent to inhibit demyelination or promote remyelination, such as for testing in an animal model as described in Example 22.

### Example 22

### In Vivo Screening of Bioactive Agents

The MRF conditional knockout mice (MRF^{fl/fl}, Olig2^{wt/cre}) as described in Example 16, are used to screen bioactive agents. The MRF conditional knockout mice (MRF^{fl/fl}, Olig2^{wt/cre}) at P11, develop tremors and seizures. In this screen, MRF conditional knockout mice are administered candidate bioactive agents prior to P11. If the animals do not develop tremors or seizures, or develop tremors or seizures that are less severe as compared to MRF conditional knockout mice that were not administered the candidate bioactive agent, the candidate bioactive agent is selected for further development as a therapeutic agent to inhibit demyelination or promote remyelination. The animal is also analyzed by immunohistochemistry, or detection of gene expression, of myelin specific genes, or by electron micrography of axons, wherein if the animals administered the candidate bioactive agent shows increased expression of myelin specific genes, or myelinated axons as compared to the animals not administered the candidate bioactive agent, the candidate bioactive agent is selected for further development as a therapeutic agent to inhibit demyelination or promote remyelination

In a second screen, MRF conditional knockout mice are administered candidate bioactive agents after the animals exhibit tremors and seizures, ie. after or about P11. If the animals' condition improves, such as a decreased extent of tremors or seizures as compared to an animal not administered the candidate bioactive agent, the candidate bioactive agent is selected for further development as a therapeutic agent to inhibit demyelination or promote remyelination. The animal is also analyzed by immunohistochemistry, or detection of gene expression, of myelin specific genes, or by electron micrography of axons, wherein if the animals administered the candidate bioactive agent shows increased expression of myelin specific genes or myelinated axons as compared to the animals not administered the candidate bioactive agent, the candidate bioactive agent is selected for further development as a therapeutic agent to inhibit demyelination or promote remyelination

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims herein define the scope of the invention and that methods and structures within the scope of these claims and their equivalents be covered thereby.

1. An isolated nucleic acid molecule comprising: a neural cell specific expression regulatory element operably linked to a nucleic acid sequence encoding MRF or a functional variant thereof.

2. (canceled)

3. The isolated nucleic acid molecule of claim 1, wherein said neural cell is a glial cell.

4. The isolated nucleic acid molecule of claim 3, wherein said glial cell is an oligodendrocyte, oligodendrocyte precursor, Schwann cell, astrocyte, or microglial cell.

5. The isolated nucleic acid molecule of claim 1, wherein said neural cell specific regulatory element is from a myelin basic protein (MBP), ceramide galactosyltransferase (CGT), oligodendrocyte-myelin glycoprotein (OMG), cyclic nucleotide phosphodiesterase (CNP), NOGO, myelin protein zero (MPZ), peripheral myelin protein 22 (PMP22), protein 2 (P2), GFAP, AQP4, PDGFα, RG5, pGlycoprotein, neurturin (NRTN), artemin (ARTN), persephin (PSPN), sulfatide or proteolipid protein (PLP), Olig1, or Olig2 gene.

6. (canceled)

7. A vector or host cell comprising said isolated nucleic acid molecule of claim 1.

8. (canceled)

9. A transgenic animal comprising a MRF transgene.

10. The transgenic animal of claim 9, wherein said MRF transgene is operably linked to a neural cell specific regulatory element.

11. (canceled)

12. (canceled)

13. The transgenic animal of claim 10, wherein said MRF transgene is flanked by recombinase sites.

14. The transgenic animal of claim 10, wherein said animal comprises a recombinase transgene.

15. The transgenic animal of claim 14, wherein said recombinase transgene is operably linked to a cell type specific regulatory element.

16. The transgenic animal of claim 14, wherein said recombinase is Cre recombinase or Flp.

17-23. (canceled)

24. A composition comprising a bioactive agent that modulates MRF activity, wherein said composition is capable of:
(a) treating a neuropathy in a subject;
(b) promoting remyelination in a subject; and/or
(c) promoting oligodendrocyte differentiation of a stem cell.

25. (canceled)

26. (canceled)

27. The composition of claim 24, further comprising a second bioactive agent, wherein said second bioactive agent induces oligodendrocyte differentiation.

28. The composition of claim 27, wherein said second bioactive agent promotes the activity of Sox10, Nxk2.2, Olig1, Olig2 or a combination thereof.

29. The composition of claim 24, wherein said neuropathy comprises demyelination.

30. The composition of claim 24, wherein said neuropathy is multiple sclerosis.

31. The composition of claim 24, wherein said bioactive agent is a peptide, antibody, aptamer, siRNA, miRNA, EGS, antisense molecule, peptidomimetic, or small molecule.

32. A method of treating a neuropathy in a subject comprising administering to said subject a therapeutically effective amount of the composition of claim 24.

33. A method of promoting remyelination in a subject comprising administering to said subject a therapeutically effective amount of the composition of claim 24.

34. A method for promoting oligodendrocyte differentiation of a stem cell comprising introducing a bioactive agent into said stem cell, wherein said bioactive agent is the composition of claim 24.

35-42. (canceled)

43. A method of screening for a candidate bioactive agent effective in modulating MRF activity comprising:
a. contacting a test cell with said candidate bioactive agent; and,
b. assaying for a change in the expression level of MRF in comparison to a control cell.

44. A method of screening for a candidate bioactive agent effective in promoting myelination and/or remyelination in an animal comprising:
a. administering a candidate bioactive agent to an animal; and,
b. assaying for an increase in the expression level of MRF in comparison to a control animal, wherein said increase is indicative of said bioactive agent promoting myelination in said animal.

45. A method of screening for a candidate bioactive agent effective in promoting remyelination and/or remyelination in an animal comprising:
a. administering a candidate bioactive agent to the animal of claim 9;
b. assaying for an increase in the expression level of at least one gene in FIG. 5, in comparison to a control animal, wherein said increase is indicative of said bioactive agent promoting myelination in said animal; and/or,
c. observing a change in myelination in said animal in comparison to said control animal.

46-60. (canceled)
